(19) United States
(12) Patent Application Publication
(10) Pub. No.: US 2003/0167500 A1
(43) Pub. Date: Sep. 4, 2003

(54) Methods and compositions for generating a genetically modified animal using lentiviral vectors

(76) Inventors: Ram Ramabhadran, Hamden, CT (US); Frank Koentgen, Canning Vale, WA (US); John K. Wakefield, Birmingham, AL (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/325,459

(22) Filed: Dec. 19, 2002

Related U.S. Application Data
(60) Provisional application No. 60/398,476, filed on Jul. 25, 2002.
(60) Provisional application No. 60/344,841, filed on Dec. 21, 2001.

(51) Int. Cl. A01K 67/027; C12N 15/867

(52) U.S. Cl. 800/21; 435/456; 800/14

(57) ABSTRACT

The present invention provides methods and compositions for the integration of a nucleotide sequence of interest into the genome. Specifically, the methods of the present invention comprise contacting a lentiviral vector with the plasma membrane of an oocyte, early stage embryo, or blastula under conditions that permit the entry of the viral vector and the subsequent integration of the nucleotide sequence of interest into the genome. The methods further comprise culturing the oocyte or embryo contacted with the lentiviral vector under conditions that allow for the formation of a pre-implantation embryo. Subsequently, the pre-implantation embryo can be transferred into a recipient vertebrate where it is allowed to develop into at least one genetically modified animal. The methods and compositions of the invention thereby allow for the production of genetically modified animals, particularly, vertebrates, and particularly mammals.

### CROSS REFERENCE TO RELATED APPLICATION

This Application claims the benefit of U.S. Provisional Application No. 60/398,476, filed Jul. 25, 2002 and U.S. Provisional Application No. 60/344,841, filed Dec. 21, 2001, both of which are herein incorporated by reference in their entirety.

### FIELD OF THE INVENTION

The present invention provides methods and compositions for the integration of a nucleotide sequence of interest into the genome of an animal.

### BACKGROUND OF THE INVENTION

The ability to manipulate the genetic make up of animals is widely used in model organisms to functionally characterize genes and to dissect complex networks for cooperating genes. Genetically modified model organisms have proven to be a promising route for gaining insight into gene functions. For instance, genetic modification in livestock has allowed for the exploration of ways to improve disease resistance, growth rate, feed efficiency, carcass composition, as well as enhance the nutritional quality and healthfulness of meat production. However, current methods for generating such genetically modified animals remain costly and inefficient.

For some time, pronuclear microinjection has been the preferred method for generating transgenic animals. In this approach, pronuclei are microinjected, resulting in the delivery of several hundred transgene copies. Of the microinjected embryos transferred to a recipient mother, 10-30% survive to term and only 1-10% of these are transgenic. Furthermore, transgenes are expressed in only about half of the genetically modified lines.

Alternatively, nuclear transfer, or cloning, is another method used to generate genetically modified animals. In these methods, transgenes are introduced into somatic cells and the cells are cultured to select those that have incorporated the new genes. The nuclei of these genes are then transferred into the cytoplasm of enucleated unfertilized eggs. While nuclear transfer ensures all animals born are transgenic, fetal survival has been very low and the technique itself is technically challenging.

Alternatively, retroviral infection, in which the genetic information is transferred as an RNA molecule, is a commonly used method for gene transfer into embryos. After entry of the retroviral RNA into the cell and reverse transcription into DNA, the integration of the DNA provirus into the host cell genome is mediated by the retroviral integrase and specific nucleotide sequences at the ends of the retroviral genome (Goff et al. (1992) Annu. Rev. Genet. 26:527-544 and Brown et al. (1989) Proc. Natl. Acad. Scie. 86:2525-2529). Most retroviruses can integrate only in dividing cells (Kulkosly et al. (1994) Parmacol. Ther. 61:185-203; Roe et al. (1993) EMBO J. 12:2099-2108, Miller et al. (1990) Mol. Cell. Biol. 10:12:4239-4242). The critical requirement for integration of retroviruses, other than HIV and other lentiviruses, is the breakdown of the nuclear envelope during mitosis. Repeated attempts over a number of years showed that the lack of control of gene dose and timing using this process results in nearly all of the animals born being genetic mosaics characterized by different gene integration locations in different tissues (Jaenisch et al. (1980) Cell 19:181-188). This necessitates outbreeding to establish homozygous and heterozygous lines suitable of analysis of gene expression.

Novel methods and compositions are needed for more efficient means for producing genetically modified animals.

### SUMMARY OF THE INVENTION

The present invention provides methods and compositions for the integration of a nucleotide sequence of interest into the genome of an animal, particularly a vertebrate, particularly a mammal. The methods of the invention comprise providing an isolated oocyte, early stage embryo, or blastula having a plasma membrane, and contacting the plasma membrane with a composition comprising an effective concentration of at least a first lentivirus vector or functional equivalent thereof comprising the nucleotide sequence of interest. Additional methods of the invention further comprise culturing the oocyte, early stage embryo, or blastula under conditions that allow formation of a pre-implantation embryo. Further methods comprise transferring the pre-implantation embryo into a recipient vertebrate and allowing the pre-implantation embryo to develop into at least one genetically modified vertebrate.

In specific methods of the invention, the early stage embryo comprises a fertilized oocyte, a 2-cell stage embryo, a 4-cell stage embryo, an 8-cell stage embryo or a morula.

Additional methods of the invention comprise contacting the plasma membrane of the isolated oocyte, early stage embryo, or blastula with a composition comprising an effective concentration of a first lentivirus vector or functional equivalent thereof comprising a first nucleotide sequence of interest and a second lentivirus vector comprising a second nucleotide sequence of interest.

In other methods of the invention, the oocyte or early stage embryo further comprises a zona pellucida, wherein the zona pellucida and the plasma membrane define a perivitelline space. In this embodiment, a composition comprising an effective concentration of at least a first lentivirus vector or functional equivalent thereof comprising the nucleotide sequence of interest is introduced into the perivitelline space. In specific embodiments, the method of introduction comprises microinjection.

Additional methods of the invention comprise introducing into the perivitelline space of the isolated oocyte or early stage embryo a composition comprising an effective concentration of a first lentivirus vector comprising a first nucleotide sequence of interest and a second lentivirus vector comprising a second nucleotide sequence of interest.

Further methods of the invention comprise the use of a lentivirus vector or functional equivalent thereof derived from a virus selected from the group consisting of human immunodeficiency viruses and simian immunodeficiency viruses. In other embodiments, the lentiviral vector is a trans-viral vector. In yet other embodiments the trans-viral vector is a trans-lentiviral vector or a trans-retroviral vector.

Compositions of the invention comprise an isolated oocyte, an early stage embryo, or blastula and an effective concentration of at least a first lentivirus vector or functional equivalent thereof comprising a nucleotide sequence of interest. Additional compositions include an oocyte or early stage embryo further comprising a zona pellucida, where the zona pellucida and the plasma membrane define a perivitelline space; and, the lentivirus vector is in the perivitelline space.

Further compositions of the invention include an isolated oocyte, early stage embryo, or blastula and an effective concentration of at least an effective concentration of a first lentivirus vector or functional equivalent thereof comprising a first nucleotide sequence of interest and an effective concentration of a second lentivirus vector or functional equivalent thereof comprising a second nucleotide sequence of interest. Additional compositions include an oocyte or early stage embryo having, in the perivitelline space, an effective concentration of a first lentiviral vector or functional equivalent thereof comprising a first nucleotide sequence of interest and an effective concentration of a second lentiviral vector or functional equivalent thereof comprising a second nucleotide sequence of interest.

In other embodiments of the invention, the lentivirus vector of the composition of the invention is derived from a virus selected from the group consisting of human immunodeficiency viruses and simian immunodeficiency viruses. In specific embodiments, the lentiviral vector is a trans-viral vector. In yet other embodiments of the invention, the trans-viral vector is a trans-lenti viral vector or a trans-retroviral vector.

### BRIEF DESCRIPTION OF DRAWINGS

FIG. 1 schematically illustrates a non-limiting example of a lentiviral vector that can be used in the methods of the present invention. Specifically, FIG. 1 provides an illustration of the “trans-lentiviral” vector design comprising four (4) different DNA constructs used to generate a trans-viral particle that can be used in the methods of the present invention. (Inactive Reverse Transcriptase and Integrase are unboxed.)

FIG. 2 provides a non-limited illustration of the trans-retroviral vector design.

FIG. 3 provides evidence that the methods and compositions of the invention have the capacity to generate multiple individual transgenic lines from each founder animal. FIG. 3A shows genomic DNA made from tail clips of offspring of one of the first eGFP founders. FIG. 3B shows a Southern blot probing for GFP. FIG. 3 provides confocal images.

### DETAILED DESCRIPTION OF INVENTION

Methods and compositions for the integration of a nucleotide sequence of interest into the genome of an animal are provided. Specifically, the methods of the present invention comprise contacting an oocyte, an early stage embryo, or a blastula with a lentiviral vector containing a nucleotide sequence of interest under conditions that permit the entry of the viral vector and the subsequent integration of the nucleotide sequence of interest into the genome. The methods further comprise culturing the unfertilized oocyte, the early stage embryo, or the blastula which was contacted with the lentiviral vector or functional equivalent thereof under conditions that allow for the formation of a pre-implantation embryo. Subsequently, the pre-implantation embryo can be transferred into a recipient animal where it is allowed to develop into at least one genetically modified animal.

The methods and compositions of the invention thereby allow for the production of genetically modified animals, particularly a vertebrate, and more particularly a mammal. Unlike other retroviruses, lentiviral vectors do not require cell division for the integration of the proviral genome into the chromosome of the target cell (Naldini et al. (1996) Science 272:263:7, herein incorporated by reference). Since the lentiviral proviral genome is capable of integrating into the genome of the oocyte or fertilized oocyte prior to the first cell division, specific embodiments of the methods of the invention allow for a reduced frequency of genetically modified animals that are mosaic for the presence of the nucleotide sequence of interest.

Moreover, as demonstrated herein, the use of the lentiviral vectors in the methods of the invention substantially increase the frequency by which genetically modified animals are produced. As the methods of the present invention allow for multiple independent integration events, a single Fo animal containing multiple integration events can be outcrossed to produced multiple founder lines having integration sites at independent locus. As such, transformation efficiencies of greater than 100% are achievable. Specifically, the methods of the invention result in a transformation efficiency of about 5% to about 10%, about 10% to about 20%, about 20% to about 60%, about 60% to about 80%, about 80% to about 100%, about 100% to about 125%, about 125% to about 175%, about 175% to about 200%, about 200% to 300% or higher. As used herein “transformation efficiency” is defined as the number of independently inheritable genetically modified animals produced divided by the total number of oocytes or embryos contacted with the lentiviral vector.

The present invention also provides methods and compositions for the introduction of multiple nucleotide sequences of interest into the genome of an unfertilized oocyte, an early stage embryo, or a blastula. In specific embodiments, the increased efficiency of DNA integration and the reduced frequency by which the mosaic animals are generated by the methods and compositions of the invention, improves the efficiency by which non-mosaic animal having integrated multiple nucleotide sequences of interest are generated.

I. Introduction of Lentiviral Vectors into Oocytes and Embryos at Various Stages of Embryonic Development

The present invention provides novel methods and compositions for the generation of a genetically modified animal. In particular, the novel methods comprise contacting an unfertilized oocyte, an early stage embryo, or a blastula stage embryo with a composition comprising an effective concentration of at least one lentiviral vector comprising a first nucleotide sequence of interest. The methods of the present invention provide an efficient means for the integration of a nucleotide sequence of interest into the unfertilized oocyte, early stage embryo or blastula and thereby provide an effective method for the production of genetically modified animals.

While the methods of the present invention can be performed in any animal, particularly the animal is a vertebrate, and more particularly the vertebrate is a mammal. In specific embodiments, the vertebrate is a human. In other embodiments the vertebrate is a non-human vertebrate. As used herein, a “non-human vertebrate” of the invention comprises all non-human vertebrates capable of producing a “genetically modified non-human vertebrate”. Such non-human vertebrates include, but are not limited to, rodents, non-human primates, ovines, bovines, ruminants, lagomorphs, porcines, caprines, equines, murines, canines, felines, aves, etc. Preferred non-human vertebrates include mice, rats, sheep, cattle, pigs, aves, and rabbits. It is further recognized that the animals encompassed by the methods and compositions of the present invention further include individual animals at all states of development, including embryonic and fetal stages.

By “genetically modified animal” or “genetically modified vertebrate” is meant any animal containing one or more cells whose genome has been altered by the integration of a nucleic acid sequence of interest. The nucleotide sequence of interest can be integrated into the genome of either a somatic and/or germ line cell of the genetically modified animal.

As used herein, “oocyte” refers to a female gamete cell and includes primary oocytes, secondary oocytes, and mature, unfertilized ovum. An oocyte is a large cell having a large nucleus (i.e., the germinal vesicle) surrounded by ooplasm. The ooplasm contains non-nuclear cytoplasmic contents including mRNA, ribosomes, mitochondria, yolk proteins, etc. The membrane of the oocyte is referred to herein as the “plasma membrane”. The mammalian oocyte is further surrounded by an extracellular envelope called the zona pellucida, to which sperm must bind and penetrate before they can make contact with the plasma membrane of the oocyte. The term “perivitelline space” refers to the space located between the zona pellucida and the plasma membrane of a mammalian oocyte and fertilized oocyte and is maintained through the early embryonic developmental stages (i.e., the zona pellucida is shed from the blastocyst prior to implantation).

As used herein an “early stage embryo” encompasses all embryonic development stages that begin upon fertilization of the oocyte (i.e., the fertilized oocyte) and extends through the 2-cell stage, the 4-cell stage, the 8-cell stage, and the morula (the 16 to 32-cell stage embryo). As defined herein, an early stage embryo does not include the blastula stage of development. As used herein, by “blastula” is intended the embryonic development stage characterized by the development of a hollow ball of cells surrounding a cavity called the blastocoel. One of skill in the art will recognize that the overall organization of the blastula will vary depending on the organism. For instance, a “blastocyst” refers to a cleavage stage mammalian embryo characterized by a hollow ball of cells made of outer trophoblast cells and inner cell mass.

By “fertilized oocyte” is intended an embryo at the “one-cell” stage of development. This stage of development is triggered by fertilization and is characterized by the second meiotic division; the extrusion of the second polar body; the formation of a paternal and maternal pronuclei; and, DNA replication. As used herein, the developmental stage encompassed by the term “fertilized oocyte” begins upon fertilization and concludes upon cleavage of the fertilized oocyte into a two-cell embryo.

As defined herein an “isolated” oocyte, an “isolated” early stage embryo (i.e., fertilized oocyte, 2-cell stage, 4-cell stage, 8-cell stage or morula), or an “isolated” blastula have been removed from the natural in vivo environment of the donor female.

A. Methods of Contacting a Lentiviral Vector to the Oocyte and Embryos of Various Developmental Stages

The present invention provides methods of integrating a nucleotide sequence of interest into the genome of a vertebrate comprising providing an unfertilized oocyte, an early stage embryo (i.e., a fertilized oocyte, a 2-cell, 4-cell, or 8-cell embryo, or a morula), or a blastula and contacting them with a composition comprising an effective concentration of at least a first lentiviral vector or functional equivalent thereof having a nucleotide sequence of interest. By “contacting” is intended the direct contact of the lentiviral vector with the oocyte, the early stage embryo, or the blastula.

In more specific embodiments, the methods of the present invention comprise contacting the plasma membrane of the oocyte, an early stage embryo (i.e., a fertilized oocyte, 2-cell, 4-cell, or 8-cell embryo, or morula) or a blastula with a composition comprising an effective concentration of at least a first lentiviral vector or functional equivalent thereof having a nucleotide sequence of interest. By “contacting with the plasma membrane” is intended the direct contact of the lentiviral vector with the plasma membrane of the oocyte, the early stage embryo or blastula. One of skill in the art will appreciate that a variety of methods may be employed to bring the viral vector and plasma membrane into direct contact.

For instance, when the oocyte or early stage embryo retains the zona pellucida, “contacting” can comprise the introduction of the lentiviral vector into the perivitelline space. In this embodiment, a nucleotide sequence of interest is integrated into the genome of a vertebrate by providing an oocyte or early stage embryo having a plasma membrane and a zona pellucida, where the zona pellucida and the plasma membrane define a perivitelline space; and, introducing into the perivitelline space a composition comprising an effective concentration of at least a first lentivirus vector comprising a first nucleotide sequence of interest under conditions that permit the entry of the viral vector into the cell.

By “introduced” is intended the use of any means known in the art to penetrate the zona pellucida and thereby allow the lentiviral vector into the perivitelline space. Methods of introducing the lentiviral vector include, for example, microinjection of the virus into the perivitelline space (Chang et al. (2001) Science 291:309-312, Chan et al. (1998) Proc. Natl. Acad. Sci. 95:14028-33 and U.S. Pat. No. 6,080,912, herein incorporated by reference); and, incubation of an unfertilized oocyte or an early stage embryo (i.e., fertilized oocyte, 2-cell, 4-cell, 8-cell, or morula) comprising the plasma membrane and the zona pellucida under conditions that allow the viral vector to enter the zona pellucida.

When microinjection techniques are employed, one of skill in the art will appreciate that the perivitelline space of most vertebrate oocytes can accommodate at least about 1-300 picoliters of injected fluid. As such, lentiviral vectors having high viral titers are preferably employed in the methods of the invention. The use of high titer stocks allows for the introduction of an effective concentration of viral particles into the perivitelline space.

The lentiviral vectors of the present invention will preferably have a stock titer of about 1×10⁶, about 1×10⁷, about 1×10⁸, about 1×10⁹, about 1×10¹⁰, about 1×10¹¹ or higher transducing units per ml. Alternatively, the lentiviral vectors of the present invention can have a viral titer ranging from about 5×10⁶ to about 5×10⁷, about 5×10⁷ to about 5×10⁸, about 5×10⁸ to about 5×10⁹, about 5×10⁹ to about 5×10¹⁰ or higher viral particles per ml. In specific embodiments, the viral titer is at least about 2.9×10⁹ transducing units per ml. One of skill will be apprised of the methods to generate a viral stock at the required concentrations.

In other embodiments of the present invention, the zona pellucida may be removed from the oocyte or early stage embryo (i.e., fertilized oocyte). In this embodiment, “contacting” can comprise incubating the composition comprising an effective concentration of the lentiviral vector with the oocyte or early stage embryo (i.e., fertilized oocyte, 2-cell, 4-cell, 8-cell or morula). Methods for the removal of the zona pellucida are known in the art. See, for example, Hogen et al. (1994) Manipulating the Mouse Embryo: A Laboratory Manual 2^{nd} Ed. Cold Spring Harbor Press. Exemplary conditions for contacting the oocyte or early stage embryo in this embodiment comprise incubating, for example, the oocyte or early stage embryo and lentiviral vector in a buffer comprising Quinns Advantage Hepes buffered medium (Sage Biofarm, order no. 1020) supplemented with 4% Bovine Serum Albumin (BSA, factor V, embryo tested, Sigma order no. A-3311) and Quinns Advantage Cleavage Medium (Sage Biofarm order no. 1026) supplemented with 4% BSA. In specific embodiments, an effective concentration comprises a viral concentration of about 1×10³, about 1×10⁴, about 1×10⁵, about 1×10⁶, about 1×10⁷ or about 1×10⁸ transduction units viral particles per μl. Alternatively, the viral particle concentration can range from about 5×10³ to about 5×10⁴, from about 5×10⁴ to about 5×10⁵, from about 5×10⁵ to about 5×10⁶, from about 5×10⁶ to about 5×10⁷, or from about 5×10⁷ to about 1×10⁸ transducing units per μl.

In other embodiments, when the embryo contacted with lentivirus is at the blastula stage of development, the method of contacting can comprise injection of the viral particle into the blastocoel cavity. See, for example, Wolfgang et al. (2001) PNAS 98:10728-10732, herein incorporated by reference.

By “effective amount” or “effective concentration” is meant a concentration of viral vector particles sufficient to contact the oocyte, early stage embryo, or blastula with at least one viral vector particle. Alternatively, the effective amount comprises contacting the oocyte, early stage embryo or blastula with about 1-10 viral particles, about 10 to about 50, about 50 to about 100, about 100 to about 1000, about 1000 to about 2000, about 2000 to about 5000, about 5000 to about 1×10⁴, 1×10⁴ to about 1×10⁵, about 1×10⁵ to about 1×10⁶ viral particles or higher.

In one embodiment of the present invention, the lentiviral stock is titered and diluted prior to microinjection into the perivitelline space so that the number of proviral genomes integrated into the resulting genetically modified animal is controlled. One of skill in the art will be able to determine the appropriate concentration of viral particle needed for the number of integration events desired.

In other embodiments of the invention, the plasma membrane is contacted with a composition comprising an effective concentration of at least two lentiviral vectors having at least two distinct nucleotide sequences of interest. As the methods of the invention minimize the production of mosaic animals, this embodiment allows for an improved efficiency for the production of genetically modified animals having integrated at least two nucleotide sequences of interest.

By “pharmaceutically acceptable carrier” is intended a carrier that is conventionally used in the art to facilitate the storage, administration, and/or the biological activity of a viral vector. A suitable carrier should be stable, i.e., incapable of reacting with other ingredients in the formulation. It should not produce significant adverse effect in recipients at the concentrations employed. Such carriers are generally known in the art. Exemplary pharmaceutically acceptable carries include, but are not limited to, sterile pyrogen-free water and sterile pyrogen-free physiological saline solution. One of skill in the art will further recognize that the pharmaceutically acceptable carrier can comprise additional compound that, for example, improve the ability of the viral particle to enter the cell. Such compounds include various cationic compounds, such as the divalent compounds polybrene and DEAE.

Methods of obtaining oocytes, early stage embryos, and blastula are known in the art. For example, fertilized oocytes can be obtained from natural matings in which the timing of ovulation and fertilization are controlled by environmental conditions rather than by administration of gonadotrophins. Alternatively, gonadotrophins can be administered to females prior to mating to increase the number of eggs that are ovulated. One of skill in the art will appreciate that the species of interest, the age and weight of the female donor, the dosage of gonadotrophins, timing of gonadotrophin administration, and the reproductive performance of the stud male will influence the specific method used to obtain fertilized oocytes. Alternatively, the oocytes can be fertilized in vitro. Such methods of oocyte isolation, their maturation, and fertilization are known. See, for example, U.S. Pat. No. 5,741,957; U.S. Pat. No. 6,080,912; Lonergan et al. (1994) Acta Vet Scand 35:307-20; Nagai et al. (2001) Theriogenology 55:1291-301 (for bovine); Moor et al. (1990) J. Reprod. Fertil Suppl 40:197-210, Nagai et al. (2001) Theriogenology 55:1291-301; Kikuchi et al. (1999) Biol Reprod 60:336-4; Funahashi et al. (1997) J. Reprod Fertil suppl 52:271-83 (for pigs); Wang et al. (2001) Reproduction 122:809-816 and Hogen et al. (1994) Manipulating the Mouse Embryo: A Laboratory Manual 2^{nd} edition, Cold Spring Harbor Press (for mice); and, Teotia et al. (2001) Small Rumin Res 40:165-177 (for caprine). All of these references are herein incorporated by reference. See also, Jackson et al. (2000) Mouse Genetics and Transgenics: A Practical Approach, Oxford University Press and Pinkert et al. (1994) Transgenic Animal Technology: A Laboratory Handbook Academic Press, San Diego, both of which are also incorporated by reference; Chan et al. (1998) PNAS 95:14028-14033; Parrish et al. (1986) Theriogenology 25:591-600; and Leibfried et al. (1979) J. Animal Science 48:76-86.

B. Methods for the Formation of a Pre-Implantation Embryo and Embryo Transfer

Following the contacting of the unfertilized oocyte, early stage embryo (i.e., fertilized oocyte, 2-cell embryo, 4-cell embryo, 8-cell embryo, or morula), or blastula with the lentiviral vector, the embryos are cultured in vitro under conditions that allow for the formation of a “pre-implantation embryo”. Such pre-implantation embryos preferably contain approximately 16 to 150 cells or more, particularly approximately 16 to 64 cells.

One of skill in the art will be apprised that the methods necessary to culture a pre-implantation embryo may vary depending on the developmental stage of the oocyte or embryo that was contacted with the lentiviral vector. For instance, when an early stage embryo or blastula is contacted, methods for culturing fertilized oocytes are known in the art and include those described by Gordon et al. (1984) Methods in Enzymology 101:414; Hogan et al. (1986) Manipulating the Mouse Embryo, Cold Spring Harbor Laboratory Press, Cold Spring Harbor, N.Y. (for the mouse embryo); Hammer et al. (1985) Nature 315:680 (for rabbit and porcine embryos); Gandolfi et al. (1987) J. Reprod. Fert. 81:23-28; Rexroad et al. (1988) J. Anim. Sci. 66: 947-953 (for ovine embryos); and Eyestone et al. (1989) J. Reprod. Fert. 85:715-720; Camous et al. (1984) J. Reprod. Fert. 72: 779-785; and Heyman et al. (1987) Theriogenology 27:5986 (for bovine embryos).

In embodiments where an unfertilized oocyte is contacted with the viral vector, formation of the pre-implantation embryo will include culturing the mature oocyte with sperm. Such methods of in vitro fertilization are known in the art. See, for example, U.S. Pat. No. 6,080,912; Chan et al. (2001) Science 291:309-312; and Chan et al. (1998) PNAS 95:14028-14033, all of which are herein incorporated by reference.

The pre-implantation embryos are thereafter transferred to an appropriate recipient female by standard methods to permit development and birth of the genetically modified animal. Interbreeding the founder genetically modified animal can then produce heterozygous and homozygous animals. Such methods are known in the art. See, for example, Hogen et al. (1994) Manipulating the Mouse Embryo: A Laboratory Manual 2^{nd} Ed. Cold Spring Harbor Laboratory Press.

The term “progeny” refers to any and all future generations derived or descending from a particular genetically modified animal, i.e., an animal having one or more nucleotide sequences of interest integrated in its genome, regardless of whether the animal is heterozygous or homozygous for the nucleotide sequence. Progeny of any successive generation which contain the nucleotide sequence of interest are included herein, such that the progeny, the F1, F2, F3 generations, and so on indefinitely containing the nucleotide sequence of interest are included in this definition.

C. Detecting the Integrated Nucleotide Sequence of Interest

A potential F₀ founder genetically modified animal (i.e., a mammal born from the oocyte or embryo contacted with the lentiviral vector) may be initially identified by techniques known in the art, including, but not limited to, PCR analysis and/or restriction enzyme digestion and/or southern blot analysis. See, Hogen et al. (1994) Manipulating the Mouse Embryo: A Laboratory Manual, 2^{nd} edition, Cold Spring Harbor Press, herein incorporated by reference. One of skill in the art will recognize that the identification of the founder genetically modified animal can occur at various stages of development, including prior to embryo transfer into the recipient female, in utero, postpartum, or after birth.

Methods to assay for the integration of the nucleotide sequence of interest in the genome are well known in the art. For instance, the integration of the nucleotide sequence of interest or an associated selectable marker can be assayed for in the pre-implantation embryo. Detection at this stage of development will allow for the identification of embryos wherein genetic modification has occurred and thereby permit the subsequent implantation of genetically modified embryos in the recipient female. In this method, one or more cells are removed from the pre-implantation embryo. Essentially, all that is required is that one of the embryos which is not analyzed for integration of the nucleotide sequence of interest, be of sufficient cell number to develop to full term in utero. Following the isolation of the sample cells from the pre-implantation embryo, analysis of the genomic DNA for the detection of the nucleotide sequence of interest may be performed. See, for example, U.S. Pat. No. 5,741,957, herein incorporated by reference.

One of skill in the art will further recognize that the detection of genetic modification in the pre-implantation embryo can be combined with embryonic cloning steps to generate a clonal population of genetically modified embryos that can be transplanted into recipient females to produce a clonal population of genetically modified animals also having the same genotype. By the same “genotype” means the genomic DNA is substantially identical between the individuals of the embryo and/or genetically modified animal population. During mitosis various somatic mutation may occur which may produce variations in the genotype of one of more cells and/or animals. Thus a population having the same genotype may demonstrate individual or subpopulation variations.

Alternatively, the integration of the nucleotide sequence of interest can be assayed at later stages of embryo development. For instance, in utero and postpartum analysis of tissue can be performed. In utero analysis is performed by several techniques, including, for example, transvaginal puncture of the amniotic cavity under echoscopic guidance. See, for example, Bowgso et al. (1975) Bet. Res. 96:124-127 and Rumsey et al. (1974) J. Anim. Sci. 39:386-391, both of which are herein incorporated by reference. The cells obtained by such methods contain genomic DNA that is subjected to PCR analysis for genomic modification. Alternately, fetal cells may be recovered by chorion puncture. This method may also be performed transvaginally and under echoscopic guidance. The chorion cells obtained are subjected to PCR analysis to determine if genetic modification has occurred.

Genetic modification can also be detected after birth. For example, a tissue biopsy such as from the ear or tail of the putative genetically modified animal can be obtained and analyzed for the presence of the genetic modification. In addition, genetic modification can also be detected by assaying for expression of the mRNA sequence, the polypeptide encoded by the nucleotide sequence of interest in a tissue, secretion (i.e., saliva) or other body fluid, or for the phenotype produced by a selectable marker contained in the DNA construct comprising the nucleotide sequence of interest.

II. Lentiviruses and Lentiviral Vectors

The present invention provides a method of integrating a nucleotide sequence of interest into the genome a mammal by contacting the plasma membrane of a fertilized oocyte with a lentiviral vector having a nucleotide sequence of interest.

As used herein, a “lentiviral vector” refers to a recombinantly modified lentivirus having a modified proviral RNA genome which comprises a nucleotide sequence of interest. As defined herein a lentiviral vector of the present invention is derived, at least in part, from the retroviral subfamily lentivirinae whose family members are characterized by the ability to infect non-dividing cells. One of skill in the art will recognize components of the lentiviral vector used in the methods of the invention may be derived from any viral source, particularly a retrovirus, more particularly a lentiviral source, so long as the resulting lentiviral vector retains the ability to integrate into non-dividing cells.

Lentiviral vectors are derived from viruses of the family Retroviridae and the subfamily lentivirinae. The Lentiviruses are associated with slow, progressive disease affecting the immune system (Coffin et al. (1997) Retroviruses, Cold Spring Harbor Laboratory Press, herein incorporated by reference) and are characterized by the ability to integrate into the genome of non-dividing cells. The lentiviruses include a variety of primate (e.g. human immunodeficiency viruses [HIV-1 and 2], and simian immunodeficiency viruses [SIV]) and non-primate viruses (e.g. maedi-visna virus [MVV], feline immunodeficiency virus [FIV], equine infectious anemia virus [EIAV], caprine arthritis encephalitis virus [CAEV] and bovine immunodeficiency virus [BIV] viruses. For a review, see for example, Romano et al. (2000) Stem Cells 18:19-39.

Many lentiviral vectors useful in the methods of the present invention are known in the art and include, HIV-1 based vectors such as those described by, but not limited to, Parolin et al. (1994) J Virol. 68: 3888-3895; Naldini et al. Science. (1996) 272: 263-267; Zufferey et al. (1997) Nat Biotechnol. 15: 871-875; Uchida et al. (1998) Proc Natl Acad Sci USA. 95: 11939-11944; Kim et al. (1998) J. Virol. 72:811-816; Poeschla et al. (1996) Proc. Natl. Acad. Science 93: 11395-11399; Mochizuki et al. (1998) J Virol. 72: 8873-8883; Gasmi et al. (1994) J Virol. 73:1828-1834; Buchschacher et al. (1992) J Virol. 66: 2731-2739; and Dropulic et al. (1996) Proc Natl Acad Sci USA. 93: 11103-11108; all of which are herein incorporated by reference. In addition, self-inactivating (SIN) lentiviral vectors have been developed and find use in the present invention. See, for example, Zufferey et al. (1998) J Virol. 72: 9873-9880 and Miyoshi et al. (1998) J Virol. 72: 8150-8157, both of which are herein incorporated by reference. HIV-2 viral vectors are also known in the art. See for example, Poeschla et al. (1996) Proc Natl Acad Sci USA. 93: 11395-11399. In addition, FIV viral vectors are known. See, for example, Johnston et al. (1999) J Virology 73:4991-5000; Johnston et al. (1999) J Virology 73:2491-2498; and Poeschla et al. (1998) Nat Med 4:354-357.

Alternatively, lentiviral vectors useful in the methods of the present invention can also comprises RNA pseudotype lentiviral vectors. Retroviral particles that package a heterologous proviral genomic RNA are referred to as RNA pseudotypes. RNA pseudotype vectors have the potential advantage or decreased risk for homologous recombination between the packaging and the transfer vectors. See, for example, Rizvi et al. (1993) J Virol. 67: 2681-2688 and Embretson et al. (1987) J Virol. 61: 2675-2683, both of which are herein incorporated by reference. For example, an HIV-1I/HIV-2 based vector has been designed by Corbeau and coworkers (Corbeau et al. (1998) Gene Ther. 5: 99-104). In addition, an HIV-1/SIV-derived vector was recently described in White et al. (1999) J Virol. 73: 2832-2840. Each of these references is herein incorporated by reference.

Other lentiviral vectors being developed include replication-defective vectors based on Caprine Arthritis Encephalitis Virus (CAEV) (Mselli-Lakhal et al. (1998) Arch Virol. 143: 681-695); equine infectious anemia virus (Mitchell et al. (1998) Lancet. 351: 346); and, bovine immunodeficiency virus. Each of these references is herein incorporated by reference.

In addition, one of skill in the art will recognize that the lentiviral vector system used in the method of the present invention can be “replication defective”. By “replication defective” is intended the viral vector viral particle is unable to reconstitute a complete viral particle in the target cell and consequently, is unable to multiply and spread to other cells.

In specific embodiment the lentiviral vector used in the methods of the invention is “infectious”. By “infectious” is intended the viral particle is able to gain entry into the target cell. In other embodiments the lentiviral vector used in the methods of the invention is capable of “transducing” the target cell. By “transducing” is intended the viral vector gains entry into the target cell and integrates the gene transfer vector into the genome of the target cell.

Methods of producing the recombinant lentiviral vectors designed for the integration of a nucleotide sequence of interest into the genome are known in the art. Briefly, lentiviral vector can be produced, for example, via a transient expression system that comprises, for example, at least three genetic elements: a gene transfer construct, a packaging construct, and an Env expression construct (Naldini et al. (1996) Science. 272, 263-267; Burns et al. (1993) Proc Natl Acad Sci USA. 90: 8033-8037; and, White et al. (1999) J Virol. 73: 2832-2840). The gene transfer construct contains retroviral cis-acting elements and the nucleotide sequence of interest. This transfer construct is contained on the modified proviral genome that is inserted into the genome of the target cell. The packaging construct directs expression of viral structural proteins, except for the envelope. Proteins expressed by the packaging construct (Gag/Pol, predominantly) form the capsid and polymerase components, and recognize specific cis-acting sequences in the retroviral RNA genome and its reverse-transcribed DNA products (Miller et al. (1997) Hum Gene Ther. 8: 803-815). This recognition leads to reverse transcription and integration. The envelope construct typically contains a heterologous envelope (e.g. vesicular stomatitis virus glycoprotein G [VSV-G]). The three expression constructs are maintained in the form of bacterial plasmids and can be transfected into mammalian cells to produce replication-defective virus stocks (White et al. (1999) J Virol. 73: 2832-2840 and Naldini et al. (1996) Science. 272: 263-267, both of which are herein incorporated by reference).

One of skill in the art will recognize that lentiviral vectors can be designed in a variety of ways and still produce a “functionally equivalent” viral particle that is useful in the methods of the present invention. A more detailed discussion of “functional equivalents” as it relates specifically to the trans-vector system is provided below. One of skill in the art will recognize from this description that the concepts apply equally to other known lentiviral vector systems.

A. Trans-Viral Vector

In one embodiment of the present invention, the lentiviral vector used comprises a “trans-viral” vector. As used herein, a lentiviral vector having a “trans-viral vector” design is characterized as separating, at least in part, nucleotide sequence encoding the Gag and the Pol polyproteins. By “polyprotein” is intended a single precursor polypeptide which is processed into individual proteins. For example, the HIV Pol polyprotein comprises Reverse Transcriptase and Integrase. The HIV Gag polyprotein comprises, for example, MA, CA, NC, and p6.

In the trans-viral vector design, the nucleotide sequence encoding the Gag-Pro-Pol polyprotein is split into at least two separate parts. The first DNA segment comprises the nucleotide sequence encoding Gag or Gag/Pro or functional equivalents thereof, and at least a second DNA segment that encodes Reverse Transcriptase and/or Integrase or function equivalents thereof. In other words, a trans-viral system is distinguishable from other viral vector systems in that the polypeptides encoding Reverse Transcriptase and Integrase are supplied in trans from at least one other DNA segment than the DNA segment encoding a functional Gag polypeptide. Consequently, the transviral vector system allows for a safer viral vector, in part, by diminishing the likelihood of generating replication competent retrovirus through genetic recombination.

In one embodiment, the trans vector design encompasses a “trans-lenti viral vector.” The “trans-lenti” viral vector design is characterized by expressing the Gag-Pro-Pol polyprotein in at least two parts: a first DNA segment that expresses Gag or Gag-Pro and at least a second DNA segment that expresses Reverse Transcriptase and/or Integrase. “Trans-lenti” viral vector design is further characterized by the use of a Vpr and/or Vpx polypeptide or a functional equivalent thereof to target the Reverse Transcriptase and Integrase to the viral particle. In this design, the Vpr and/or Vpx polypeptides are used as vehicles to deliver functional Reverse Transcriptase and Integrase into the viral particle. Further details regarding the design of the Vpr/Vpx fusion proteins used in the trans-lenti viral vector design are outlined below.

In yet another embodiment, the trans-vector design encompasses a “trans-retroviral vector” design. As explained in further detail below, the “trans-retroviral” vector is characterized by expressing the Gag-Pro-Pol polyprotein in at least two parts: a first DNA segment that expresses Gag or Gag-Pro and at least a second DNA segment that expresses Reverse Transcriptase and/or Integrase. The “trans-retroviral” vector design is further characterized by the use of at least a fragment of the Gag polypeptide that is capable of being targeted to the viral particle, or a functional equivalent thereof, to target the Reverse Transcriptase and Integrase to the viral particle. In this design, the fragment of the Gag polypeptide is used as vehicles to deliver functional Reverse Transcriptase and Integrase into the viral particle. Further details regarding the design of the trans-retro viral vector design are outlined below.

One of skill will recognize that the trans-retroviral vector design can be used in viral vectors derived from any retroviral source. Accordingly, in specific embodiments of the present invention, a trans-retroviral vector is derived from a retrovirus other than a lentivirus. Such retroviral vectors can be derived from, but not limited to, retrovirus, including but not limited to, Moloney Leukemia Virus (MLV), Abelson murine leukemia virus, AKR (endogenous) murine leukemia virus, Avian carcinoma, Mill Hill virus 2, Avian Leukosis virus—RSA, Avian myeloblastosis virus, Avian myelocytomatosis virus 29, Bovine syncytial virus, Caprine arthritis encephalitis virus, Chick syncytial virus, Equine infectious anemia virus, Feline leukemia virus, Feline syncytial virus, Finkel-Biskis-Jinkins murine sarcoma virus, Friend murine leukemia virus, Fujinami sarcoma virus, Gardner-Arnstein feline sarcoma virus, Gibbon ape leukemia virus, Guinea pig type C oncovirus, Hardy-Zuckerman feline sarcoma virus, Harvey murine sarcoma virus, Human foamy virus, Human spumavirus, Human T-lymphotropic virus 1, Human T-lymphototropic virus 2, Jaagsiekte virus, Kirsten murine sarcoma virus, Langur virus, Mason-Pfizer monkey virus, Moloney murine sarcoma virus, Mouse mammary tumor virus, Ovine pulmonary adenocarcinoma virus, Porcine type C oncovirus, Reticuloendotheliosis virus, Rous sarcoma virus, Simian foamy virus, Simian sarcoma virus, Simian T-lymphotropic virus, Simian type D virus 1, Snyder-Theilen feline sarcoma virus, Squirrel monkey retrovirus, Trager duck spleen necrosis virus, UR2 sarcoma virus, Viper retrovirus, Visna/maedi virus, Woolly monkey sarcoma virus, and Y73 sarcoma virus human-, simian-, feline-, and bovine immunodeficiency viruses (HIV, SIV, FIV, BIV). See also, U.S. patent application Ser. No. 09/578,548.

A complete description of the “trans” viral vector design, the “trans-lenti” viral vector design, and the “trans-retroviral” vector design and the viral vectors systems used to produce these viral particles has been described in detail in U.S. patent application Ser. Nos. 09/089,900; 09/709,751; 09/460,548; U.S. Pat. No. 6,001,985; PCT Patent Application No. PCT/US00/18597, in Wu et al. (1997) EMBO 16:5113-5122; all of which are herein incorporated by reference.

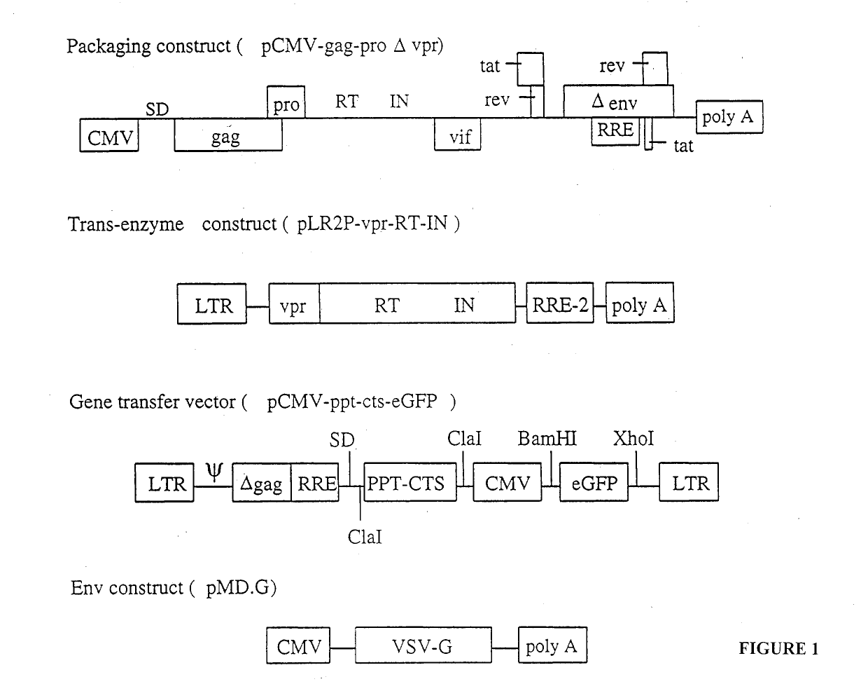

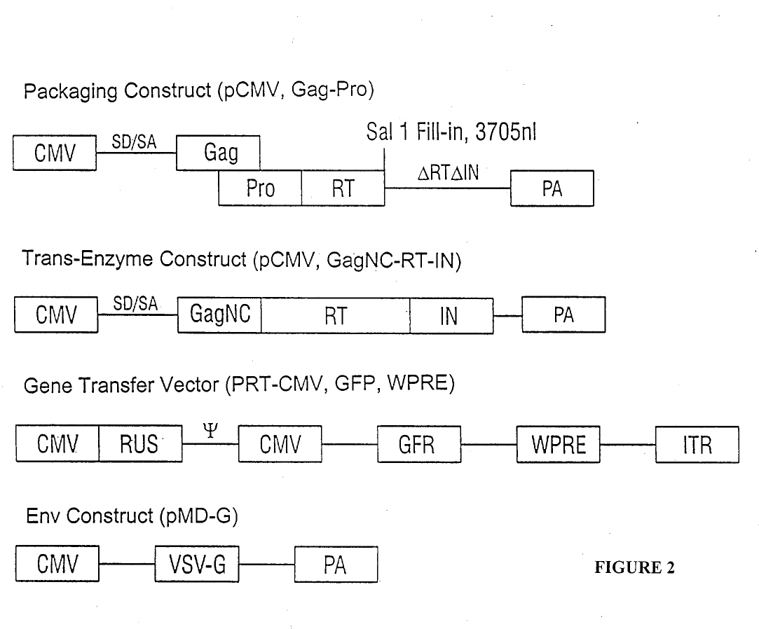

A non-limiting illustration of the trans viral vector system used in the methods of the present invention is provided in FIGS. 1-2. In these examples, the trans-vector system comprises the following components: an env construct, a packaging construct, a trans-enzyme construct, and a retroviral gene transfer vector. The “packaging construct” of the trans-viral system comprises a nucleotide sequence encoding Gag/Pro (represented as boxed structures in FIGS. 1-2, while the nucleotide sequences encoding Reverse Transcriptase (RT) and Integrase (IN) have been either deleted completely from the construct or disrupted in some manner that prevents the expression of a functional polypeptide. The nucleotide sequences encoding the Reverse Transcriptase and Integrase polypeptides are provided in trans to the packaging construct on a stretch of DNA referred to herein as the “trans-enzyme construct”. The viral expression system thereby disarms the Gag-Pro-Pol structure by splitting Gag-Pro from the nucleotide sequences encoding Reverse Transcriptase and Integrase.

The trans-viral vectors produced by the trans-viral system can be distinguished physically from viral vectors that use a three-vector system where the Gag/Pol is expressed as a polyprotein. See, for example, Wu et al. (1997) EMBO J 16:5113-5122 and Wu et al. (2000) Mol. Therapy 1:47-55, which provide assays to identify uncleaved Vpr/Vpx fusion proteins in the trans-lentivirus particles and assays that measure a reduced level of genetic recombination in the trans-viral vector when compared to the three vector lentiviral vectors.

As used herein “nucleic acid sequences” will sometimes be used as a generic term encompassing both DNA and RNA fragments. As the materials of the invention include modified retroviral genomes and their proviral counterparts, particular functional sequences referred to will occur both in RNA and DNA form. The corresponding loci will be referred to interchangeably for their occurrences in both DNA and RNA. For example, the ψ packaging signal functions in the retroviral RNA genome as a packaging signal; however, the corresponding sequences occur in the proviral DNA. Similarly, promoter, enhancer, and terminator sequences occur, though in slightly different forms, in both the genomic RNA and proviral DNA forms. The interchangeability of these functionalities in the various phases of the viral life cycle is understood by those in the art, and accordingly, rather loose terminology in regard to DNA or RNA status is often used in referring to them. Specifically, sequences specified by a progression of bases should be understood to include these specific sequences and their complements, both in DNA and RNA forms.

While a description of the various elements of the trans viral vector system, including, for example, the components used on the gene transfer vector, the packaging construct, the envelope construct, and the trans-enzyme construct of the trans-viral vector system are provided below, it is recognized that one of skill in the art can readily generate “functionally equivalent” constructs. By “functionally equivalent” construct is intended each DNA construct (i.e., the packaging construct, the gene transfer vector, the envelope construct, and the trans-enzyme construct) have substantially the same function as the specific vector constructions illustrated herein. It is further recognized that the genetic elements in the various vectors of the trans-viral vector system may be from any viral source, particularly a retrovirus, and more particularly a lentiviral source.

Examples and assays for the functional equivalence of the various components of the trans-vector system are described more fully below. While, table 1 provides a reference for various genetic elements of the HIV-1 genome and is based on NCBI Genbank Accession Number AF033819, it is recognized that sequences from other retroviruses and/or lentiviruses are known in the art and can be used to construct functionally equivalent vectors and vector systems directed to a given host species of animal. A more detailed explanation of the components outlined in Table 1 and their function may be found in Coffin et al. (1997) Retroviruses, Cold Spring Harbor Laboratory Press, New York, herein incorporated by reference. Moreover, those of skill will appreciate that allelic variations in the various genetic elements exist between different isolates of the viruses and such variants may be used in the constructs of the present invention. For instance, such viral isolates are described in Li et al. (1992) J. Virol. 66:6587; Ghosh et al. (1993) Virology 194:858; and, U.S. Pat. No. 5,869,313; all of which are herein incorporated by reference.

TABLE 1
Genetic Elements and Coordinates of a Human HIV-1 Isolate

| Genetic Element | Coordinates |
|---|---|
| R | (1-96) |
| U5 | (97-181) |
| PBS | (182-199) |
| gag | (336-1836) |
| pro | (1637-2099) |
| pol | (2102-4640) |
| vif | (4587-5163) |
| vpr | (5105-5339) |
| tat | (5377-5591, 7925-7968) |
| rev | (5516-5591, 7925-8197) |
| vpu | (5608-5854) |
| env | (5771-8339) |
| nef | (8343-8710) |
| PPT | (8615-8630) |
| U3 | (8631-9085) |
| R | (9086-9181) |

Functionally equivalent sequences of the present invention also encompass various fragments of a retroviral genome that retain substantially the same function as the respective native sequence. Such fragments will comprise at least about 10, 15 contiguous nucleotides, at least about 20 contiguous nucleotides, at least about 24, 50, 60, 80, 100, 120, 140, 160, 180, 200, 220, 240, 260, 280, 300, 340, 360, 380, or up to the entire contiguous nucleotides of the specific genetic element of interest. Such fragments may be obtained by use of restriction enzymes to cleave the native viral genome; by synthesizing a nucleotide sequence from the native nucleotide sequence of the virus genome; or may be obtained through the use of PCR technology. See particularly Mullis et al. (1987) Methods Enzymol. 155:335-350, and Erlich, ed. (1989) PCR Technology (Stockton Press, New York). Again, variants of the various vector components, such as those resulting from site-directed mutagenesis, are encompassed by the methods of the present invention. As described in more detail below, methods are available in the art for determining functional equivalence.

By “variant” is intended substantially similar sequences. Thus, for nucleotide sequences or amino acid sequences, variants include sequences that are functionally equivalent to the various components of the lentiviral vector system. Variant nucleotide sequences also include synthetically derived nucleotide sequences that have been generated, for example, by site directed mutagenesis but which still retain the function of the native sequence. Generally, nucleotide sequence variants or amino acid sequence variants of the invention will have at least 70%, generally 80%, 85%, 90%, 91%, 92%, 93%, 94%, 95%, 96%, 97%, 98%, or 99% sequence identity to its respective native nucleotide sequence.

Variants of the nucleotide sequences can encode amino acid sequences that differ conservatively because of the degeneracy of the genetic code. These naturally occurring allelic variants can be identified with the use of well-known molecular biology techniques, such as polymerase chain reaction (PCR) and hybridization techniques. Variant nucleotide sequences also include synthetically derived nucleotide sequences that have been generated, for example, by using site-directed mutagenesis, but which still remain functionally equivalent.

With respect to the amino acid sequences for the various full length or mature polypeptides used in the lentiviral vector system used in, the present invention, variants include those polypeptides that are derived from the native polypeptides by deletion (so-called truncation) or addition of one or more amino acids to the N-terminal and/or C-terminal end of the native polypeptide; deletion or addition of one or more amino acids at one or more sites in the native polypeptide; or substitution of one or more amino acids at one or more sites in the native polypeptide. Such variants may result from, for example, genetic polymorphism or from human manipulation. Methods for such manipulations are generally known in the art.

For example, amino acid sequence variants of a polypeptide can be prepared by mutations in the cloned DNA sequence encoding the specific vector element of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (2d ed., Cold Spring Harbor Laboratory Press, Plainview, N.Y.); U.S. Pat. No. 4,873,192; and the references cited therein; herein incorporated by reference. Guidance as to appropriate amino acid substitutions that may not affect biological activity of the various vector polypeptide may be found in the model of Dayhoff et al. (1978) Atlas of Polypeptide Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

A variant of a native nucleotide sequence or native polypeptide has substantial identity to the native sequence or native polypeptide. A variant may differ by as few as 1 to 10 amino acid residues, such as 6-10, as few as 5, as few as 4, 3, 2, or even 1 amino acid residue. A variant of a nucleotide sequence may differ by as low as 1 to 30 nucleotides, such as 6 to 20, as low as 5, as few as 4, 3, 2, or even 1 nucleotide residue.

By “sequence identity” is intended the same nucleotides or amino acid residues are found within the variant sequence and a reference sequence when a specified, contiguous segment of the nucleotide sequence or amino acid sequence of the variant is aligned and compared to the nucleotide sequence or amino acid sequence of the reference sequence. Methods for sequence alignment and for determining identity between sequences are well known in the art. With respect to optimal alignment of two nucleotide sequences, the contiguous segment of the variant nucleotide sequence may have additional nucleotides or deleted nucleotides with respect to the reference nucleotide sequence. Likewise, for purposes of optimal alignment of two amino acid sequences, the contiguous segment of the variant amino acid sequence may have additional amino acid residues or deleted amino acid residues with respect to the reference amino acid sequence. The contiguous segment used for comparison to the reference nucleotide sequence or reference amino acid sequence will comprise at least 20 contiguous nucleotides, or amino acid residues, and may be 30, 40, 50, 100, or more nucleotides or amino acid residues. Corrections for increased sequence identity associated with inclusion of gaps in the variant's nucleotide sequence or amino acid sequence can be made by assigning gap penalties. Methods of sequence alignment are well known in the art.

The determination of percent identity between two sequences can be accomplished using a mathematical algorithm. For example, percent identity of an amino acid sequence can be determined using the Smith-Waterman homology search algorithm using an affine 6 gap search with a gap open penalty of 12 and a gap extension penalty of 2, BLOSUM matrix 62. Alternatively, percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic Version G. The Smith-Waterman homology search algorithm is taught in Smith and Waterman (1981) Adv. Appl. Math 2:482-489, herein incorporated by reference. Alternatively, the alignment program GCG Gap (Wisconsin Genetic Computing Group, Suite Version 10.1) using the default parameters may be used. The GCG Gap program applies the Needleman and Wunch algorithm and for the alignment of nucleotide sequences with an open gap penalty of 3 and an extend gap penalty of 1 may be used. Another preferred, non-limiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences having sufficient sequence identity. BLAST protein searches can be performed with the XBLAST program, score=50, wordlength=3, to obtain amino acid sequences having sufficient sequence identity. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

Within preferred embodiments of the invention, the DNA constructs described below are contained on a vector or plasmid. The plasmid may contain a bacterial origin of replication, one or more selectable markers, a signal that allows the plasmid construct to exist single stranded (i.e., a M13 origin of replication), a multiple cloning site, and a “marnmalian” origin of replication (i.e., a SV40 or adenovirus origin of replication). Such vectors are known in the art.

1. Gene Transfer Vector

As noted above, the trans-viral vector system provides a gene transfer vector which in combination with the packaging construct, the env construct, and the trans-enzyme construct, which enable the construction of a trans-viral packaging cell line which precludes the formation of a replication competent virus.

As used herein the “gene transfer vector” refers to a nucleotide sequence that has the necessary “cis acting” components that allow for the transcription of the gene transfer vector; encapsudation of the gene transfer vector mRNA (i.e., modified proviral genome) into the viral particle; reverse transcription of the gene transfer vector mRNA; and integration of the gene transfer vector into the genome of the target cell.

Cis acting nucleic acids sequences that carry out the functions described above are well know in the art. For instance, a gene transfer vector can comprise the following components: a 5′ LTR; a packaging signal; a Rev Responsive Element (RRE); and a 3′ LTR or any functional variant or derivative of each of these elements. The gene transfer vector can further comprise at least one nucleotide sequence of interest operably linked to a promoter active in the desired target cell. Each of these components is described in more detail below.

The 5′ and 3′ LTR sequences flank the other elements of the gene transfer vector. The LTR sequences contain multiple elements including, for example, promoter/enhancer elements along with other cis-acting sequence elements important for integration and integration of the proviral genome into the genome of the target cell. Various cis-acting elements of the LTR include, for example, the U5 region (nt 97-181 of GenBank Accession No. AF033819) and the U3 region (nt 8631-9085 of GenBank Accession No. AF033819) which comprises viral promoter and enhancer sequences that direct the expression of the retroviral gene transfer vector into a single precursor mRNA. Other LTR components include the R region which comprises sequences required for RNA transcription initiation (i.e., the transactivating region (TAR)), the polyadenylation signals (nt 1-96 of GenBank Accession No. AF033819). A more complete description of LTR sequences and functional variants for the HIV-1 virus can be found in Pereira et al. (2000) Nucleic Acid Research 28:663-668, herein incorporated by reference. One of skill in the art will recognized that the various components of the gene transfer vector can be arranged in any order as long as they are internal to the 5′ and 3′ LTR elements. The transfer vector can further comprise tRNA primer binding site sequences (nt 182-199 of GenBank Accession No. AF033819 which functions in the initiation of reverse transcription. Such alterations are known to one of skill in the art. It is further recognized that modifications to the LTRs can be made, such as those of the self-inactivating (SIN) vectors. Such alterations are known to one of skill in the art.

As used herein the “packaging signal” or “ψ signal” refers to a nucleic acid sequence that is required in cis for the encapsudation of the viral RNA into the viral particle. The packaging signal used in the methods of the present invention may be a minimal packaging signal required for encapsudation of the gene transfer vector into the viral particle. This minimal packaging sequence for the preferred retroviral gene transfer vector of the present invention will be sufficient to direct the incorporation of the modified proviral genome (i.e., gene transfer vector) into the viral particle.

It is recognized that variants or fragments of known packaging signals may be used in the methods of the present invention so long as the variants direct the encapsudation of the retroviral gene transfer vector into the viral particle. For instance, extended packaging signals which encompass sequences surrounding the minimal packaging sequence may increase the efficiency of encapsudation of the gene transfer vector in the viral particle. The HIV packaging signal has been further characterized in Mcbirde et al. (1997) J. Virol. 71:4544-4554, which is herein incorporated by reference.

The gene transfer vector may also contain a Rev Responsive Element (RRE). The presence of this element allows Rev to direct the nuclear export of the RRE-containing mRNAs. The sequence of the RRE and functional variants thereof are known in the art. See, for example, Berchtold et al. (1995) Virology 211:285-289; Dillon et al. (1990) J. Virol. 64:4428-4437; Le et al. (1990) Nucleic Acid Research 18:1613-1623; all of which are herein incorporated by reference. It is further recognized that Rev/RRE can be substituted with other elements, including for example, the cis-acting 219-nucleotide constitutive transport element (CTE) from the Mason-Pfizer monkey virus (MPMV) that has been shown to allow Rev-independent HIV-1 replication. See, for example, Bray et al. (1994) Proc. Natl. Acad Sci USA 91:1256-1260.

The gene transfer vector further comprises a DNA construct having a nucleotide sequence of interest. Thus, the nucleotide sequence of interest contained in the gene transfer vector, when introduced into the target cell via the viral particle, can be expressed. The nucleotide sequence of interest and the DNA construct comprising this sequence are describe more fully below. It is further recognized that the gene transfer vector may contain at least one additional nucleotide sequence of interest.

The gene transfer vector may further contain at least one DNA construct comprising a selectable marker operably linked to a promoter. Details regarding the use of selectable markers are describe more fully below. One of skill will appreciate that numerous possibilities exist. It is further recognized that the promoter selected for the expression of selectable marker will vary depending on if the marker is being used to monitor incorporation of the gene transfer vector into the packaging cell line or into the genome of the target cell.

FIGS. 1-2 illustrate non-limiting examples of a gene transfer vector from the trans-viral system. The construct illustrated comprises the LTR sequence from HIV-1 flanking the following components: a packaging signal; an RRE; a central polypurine tract (PPT) and a nucleotide sequence of interest operably linked to the CMV promoter.

2. Trans-Enzyme Construct

The trans-enzyme construct contains the nucleotide sequences encoding Reverse Transcriptase and Integrase apart from their native configuration. In particular, the trans-enzyme construct encodes a fusion protein comprising a first polypeptide characterized by the ability to be targeted to a viral particle, operably linked to functional Reverse Transcriptase and/or Integrase polypeptide. For instance, an HIV virion-associated accessory protein (Vpr or Vpx) or a variant or fragment thereof can be used as a vehicle to deliver polypeptides having integrase activity and reverse transcriptase activity into the trans-viral vector particle. This “trans-lenti” viral vector design is illustrated in FIG. 1. Specifically, the trans-enzyme construct may comprise a nucleic acid sequence encoding a fusion protein comprising a Vpr or Vpx polypeptide or functional variant or fragment thereof, fused in frame to at least one heterologous polypeptide comprising Integrase and/or Reverse Transcriptase or functional variants and fragments thereof. Such trans-enzyme constructs are know in the art. See, for example, Liu et al. (1997) J. Virol. 71:7704-7710, Wu et al. (1997) EMBO J. 16:5113-5122; U.S. Pat. No. 6,001,985; U.S. patent application Ser. No. 09/089,900 filed Jun. 3, 1998; and, U.S. application Ser. No. 09/460,548 filed Dec. 14, 1999; all of which are herein incorporated by reference.

As used herein, by “fusion protein” is intended a polypeptide having at least two heterologous polypeptide sequences joined for in-frame expression. That is, the nucleotide sequences encoding the heterologous polypeptides will be translated into a single translation product. In one embodiment, the fusion protein encoded by the trans-enzyme construct comprises a Vpr or a Vpx polypeptide or a functional fragment or variant thereof, while the second polypeptide comprises a polypeptide having integrase or reverse transcriptase activity. In other embodiments of the present invention, the fusion protein encoded by the trans-enzyme construct comprises nucleotide sequences encoding all three polypeptides (i.e., Vpr/Vpx, Reverse Transcriptase, and Integrase).

Sequences encoding Vpr and Vpx polypeptides are known in the art. See, for example, U.S. Pat. No. 5,861,161, herein incorporated by reference. Fragments and variants of a Vpr or Vpx polypeptide can be used and will retain the ability to be incorporated into virion particles. Examples of fragments and variants of the Vpr/Vpx polypeptides that retain this activity are known. See, for example, Paxton et al. (1993) J. Virol. 67:7229-7237 and U.S. Pat. No. 6,043,081, both of which are herein incorporated by reference. In addition, assays to determine if a Vpr or Vpx polypeptide are incorporated into a virion are routine in the art. Briefly, a fragment or variant of a Vpr/Vpx polypeptide fused to a marker polypeptide is expressed in a packaging cell line capable of producing viral particles. The cell line is cultured and viral particles are produced. The viral particles are isolated and assayed for the presence of the marker protein. See, for example, Paxton et al. (1993) J. Virol. 67:7229-7237.

Another embodiment of the trans-vector design is illustrated in FIG. 2. The design in this particular non-limited example is referred to herein as the trans-retoviral vector design which uses a fragment of the Gag polypeptide which retains the ability to be targeted to the viral particle to deliver the polypeptides having integrase activity and reverse transcriptase activity into the trans-viral vector particle.

Specifically, in this embodiment, the trans-enzyme construct may comprise a nucleic acid sequence encoding a fusion protein comprising a fragment of a Gag polypeptide which retains the ability to be targeted to the viral particle, fused in frame to at least one heterologous polypeptide comprising Integrase and/or Reverse Transcriptase or functional variants and fragments thereof. Such trans-enzyme constructs are know in the art. See, for example, U.S. patent application Ser. No. 09/578,548, filed Dec. 14, 1999.

In one embodiment, the fusion protein encoded by the trans-enzyme construct comprises a fragment of a Gag which retains the ability to be targeted to the viral particle, while the second polypeptide comprises a polypeptide having integrase or reverse transcriptase activity. In other embodiments of the present invention, the fusion protein encoded by the trans-enzyme construct comprises nucleotide sequences encoding all three polypeptides (i.e., Gag:Reverse Transcriptase: Integrase).

Sequences encoding Gag polypeptides are known in the art. Fragments and variants of the Gag polypeptide will retain the ability to be incorporated into virion particles. Examples of fragments and variants of the Gag polypeptides that retain this activity are known.

Nucleotide sequences encoding Integrase polypeptides are also known (see Table 1). Integrase is involved in many aspects of the viral life cycle. For instance, Integrase has been shown to be involved in various steps of the virion assembly and maturation process and is involved in reverse transcription and integration of the viral genome in the host cell (Lie et al. (1999) J. Virol. 73:8831-8836 and Craigie et al. (2001) J. Bio. Chem. Manuscript R100027200). The Integrase polypeptide used in the methods of the present invention may be from any viral source, particularly a retrovirus, particularly a lentiviral source.

The Integrase polypeptide or variants or fragments thereof will have integrase activity. By integrase activity is intended the polypeptide retains sufficient activity to support the production of a viral particle (i.e., supports virus assembly, maturation, and/or integration). The regions of the Integrase polypeptide that influence virus assembly, maturation, and integration are known in the art, as are assays to determine these functions. For instance, mutations in the catalytic center of Integrase decreases infectivity of the viral particle. See, for example, Liu et al. (1997) J. Virol. 71:7704-7710; Wu et al. (2000) Mol. Therapy 2:47-55; and Craigie et al. (2001) J. Bio. Chem. Manuscript R100027200 and Liu et al.(1999) J. Virol. 23:8831-8836; all of which are herein incorporated by reference.

Nucleotide sequences encoding Reverse Transcriptase are known in the art (see Table 1). Reverse Transcriptase is involved in the synthesis of double stranded, linear DNA from a single-stranded RNA template using cellular tRNA as a primer. The Reverse Transcriptase used in the present invention may be from any retroviral source, particularly a lentivirus including, but not limited to, HIV-1, HIV-2, and SIV. The Reverse Transcriptase polypeptide or variant or fragment thereof will have reverse transcriptase activity.

By “reverse transcriptase activity” is intended the polypeptide retains sufficient activity to support the production of a viral particle (i.e., catalyze replication of the gene transfer vector). Assays to measure reverse transcriptase activity are known in the art. For example, measurements of reverse transcriptase activity can be carried out in-vitro using an artificial template/primer construct and tritiated deoxynucleotide triphosphate as the nucleotide substrate. This assay system is based on detecting incorporation of radioactivity in RNA/DNA hybrids which can be precipitated with trichloroacetic acid (TCA). Other methods for determining reverse transcriptase activity can be found in, for example, U.S. Pat. No. 6,132,995 herein incorporated by reference.

It is well within skill in the art to generate an expression vector having at least two nucleotide sequence encoding heterologous polypeptides that will be translated into a single translation product (i.e., fused in frame). Furthermore, one of skill in the art will recognize that the linkers may be placed between the nucleotides sequences that encode the heterologous polypeptides of the trans-enzyme construct, so long as the linker sequence allows the coding regions of the peptides to remain in frame. Such linker sequences include, for example, protease cleavage sites that are recognized by the protease encoded on the packaging construct. Such sequences are known in the art and include, for example, the 33 nucleotides 5′ to the Reverse Transcriptase coding sequence of the HIV-1 genome or alternatively, the 18 nucleotides located 5′ to the Integrase coding sequence of GenBank Accession No. LO2317. See, for example, Wu et al. (1997) EMBO J. 16:5113-5122. One of skill in the art will recognize how to use such sequences to result in effective cleavage of the Reverse Transcriptase or Integrase from the Vpr or Vpx polypeptide.

To produce a replication defective trans-viral particle both Integrase and Reverse Transcriptase are expressed in the packaging cell line in trans to the packaging construct which encodes the Gag or Gag/Pro polypeptide. In one embodiment of the present invention, fusion proteins comprising the Reverse Transcriptase and Integrase are expressed in the packaging cell lines on two separate trans-enzyme constructs. In this embodiment, the packaging cell line will contain at least two trans-enzyme constructs. Using the trans-lentiviral vector design as an example, the first fusion protein comprising Vpx or Vpr and Reverse Transcriptase, and the second trans-enzyme construct encoding a fusion protein comprising Vpx or Vpr and Integrase. It is recognized that the order of the polypeptides in these constructs may vary. Moreover, it further recognize that the trans-retroviral vector design can be used in a similar manner.

In another embodiment of the trans-enzyme construct, the polypeptide encoding the Reverse Transcriptase and Integrase or variants or fragments thereof are expressed as a single translation product. In this embodiment, the trans-enzyme construct encodes a fusion protein having the following polypeptides or functional variants or fragments thereof fused in frame: Vpr or Vpx, Reverse Transcriptase, and Integrase. See, for example, U.S. patent application Ser. No. 09/089,900 and Wu et al. (1997) EMBO J. 16:5113-5122.

The nucleotide sequence encoding the fusion protein of the trans-enzyme construct is operably linked to a promoter active in the packaging cell line. The DNA construct containing the trans-enzyme construct may further comprise transcriptional and translational termination regions which are also functional in the packaging cell line. Promoters of interest include, for example, the chicken beta actin promoter CMV, HIV-2 LTR, the SHVTK promoter, the RSV promoter, the adenovirus major late promoter and the SV 40 promoters.

FIG. 1 illustrates one non-limiting example of a trans-enzyme construct of the trans-lentiviral vector system. The construct comprises the following operably linked components: a CMV promoter; a nucleotide sequence encoding a Vpr polypeptide; a nucleotide sequence encoding Reverse Transcriptase; a nucleotide sequence encoding Integrase; the RRE from HIV-2; and a SV40 polyadenylation signal. The trans-enzyme construct illustrated in FIG. 1, preserves the N-terminal protease cleavage site of Reverse Transcriptase and the protease cleavage site between the Reverse Transcriptase and Integrase polypeptides.

3. Packaging Construct

As noted above, the trans-viral particle used in the methods of the present invention further provides a packaging construct, which in combination with the gene transfer vector, the env construct, and the trans-enzyme construct, enable the construction of a packaging cell line which precludes the formation of a replication competent virus.

The packaging construct is characterized as a nucleic acid sequence comprising at least one nucleotide sequence that encodes a truncated Gag/Pol sequence (i.e., Gag/Pro) which does not encode a functional Integrase or Reverse Transcriptase polypeptide. A nucleotide sequence that encodes a Gag/Pro polypeptide comprises a variety of structural proteins that make up the core matrix and nucleocapsid polypeptides. The sequence further encodes a functional protease. The Gag/Pro sequences may be derived from any retrovirus as described elsewhere, herein. Such Gag/Pro sequences are known in the art and include, for example, nucleotides 336-2099 of Genbank Accession No. AF033819.

The packaging construct can further contain nucleotide sequences from a viral genome (particularly the retroviral genome) which are necessary for the production of a replication defective viral particle. Examples of genetic elements that may be contained in the packaging construct include, but are not limited to, nucleotide sequences encoding Vif, Tat, and Rev. The packaging construct, however, does not contain nucleotide sequences which encode a functional Envelope polypeptide, a functional Reverse Transcriptase polypeptide, and a functional Integrase polypeptide. These sequences have been totally or partially deleted or alternatively, have been altered to prevent translation of a functional polypeptide. Furthermore, the packaging construct lacks a functional packaging signal (ψ signal) thereby preventing the RNA produced from this construct from being incorporated into the viral particle.

As explained in more detail in U.S. patent application Ser. No. 09/089,900, the manner in which the Reverse Transcription and Integrase are mutated in the packaging construct may affect the infectivity of the viral particle. It is recognized that any alteration can be made to the reverse transcriptase and/or integrase sequence in the packaging construct that disrupts the function of the polypeptides and still allows for the production of an infectious, replication defective trans-viral vector when the Reverse Transcriptase and Integrase are express in trans in the trans-enzyme construct. For instance, in one embodiment, the reverse transcriptase and integrase sequences are altered to contain a stop codon 3′ to the protease sequence. It is further recognized that multiple mutations may be introduced into the reverse transcriptase and integrase sequence. For example, in addition to introducing a translation stop early in the coding sequence of Reverse Transcriptase, at least one additional “fatal” mutation can be positioned within the Reverse Transcriptase and/or Integrase coding sequence. This additional mutation further decreases the likelihood of reestablishing a complete Gag-Pol coding region by genetic recombination between the packaging construct and the trans-enzyme construct.

The nucleotide sequences of the packaging construct are contained in a DNA construct which further comprises a promoter active in the packaging cell line. The DNA construct may further comprise transcriptional and translational termination regions which are also functional in the packaging cell line. Promoters of interest include, for example, CMV, HIV-2 LTR, the HCMV-IE (Naldini et al. (1996) Science 272:263-267), the SHVTK promoter, the RSV promoter, the adenovirus major late promoter and, the SV40 promoters. The packaging construct may further contain a selectable marker operably linked to an active promoter.

One of skill in the art will recognize that various functional variants of the packaging construct can be envisioned. FIGS. 1-2 illustrate a non-limiting example of a packaging construct useful in the methods of the present invention. The construct comprises a CMV promoter operably linked to a nucleotide sequence encoding Gag/Pro, Vif, Tat, Rev (nt 258-8384 of Genbank Accession No. L02317). The packaging construct of FIG. 1 further comprises translational stop codons (TAA) at the first amino acid position of the Reverse Transcriptase and Integrase coding sequences, a deletion of the ψ signal, a frame shift mutation in the Vpr coding sequence, a complete deletion of the nef gene, an internal deletion that results in an inactive Vpu polypeptide, and a deletion of the nucleotides which encode the Env polypeptide. For a more detailed description of the construction of the packaging construct see U.S. patent application Ser. No. 09/089,900.

4. Env Construct

The present invention further provides an env construct which in combination with the packaging construct, gene transfer vector, and trans-enzyme construct described above, preclude formation of a replication complete trans-virus vector particle.

The env construct of the trans-viral system comprises a nucleotide sequence encoding an envelope protein or a functional variant or fragment thereof operably linked to an active promoter. A variety of envelope polypeptides are known in the art. It is recognized that the host range of cells that the viral particles of the present invention can infect can be altered depending on the envelope coding sequence used. Viral envelope proteins useful in the present invention include HIV envelope polypeptides (see Table 1), the MLV envelope glycoprotein (Page et al. (1990) J. Virol. 64:5270-5276), the vesicular stomatitis virus G-protein (VSV-G) (Yee et al. (1994) Proc. Natl. Acad. Sci. 91:9564-9568 and Burns et al. (1993) Proc. Natl. Acad. Sci. USA 90:8033-8037) or the envelope polypeptides of Ebola and Makola (Kobinger et al. (2001) Nature Biotechnology 19: 225-230). In preferred embodiments of the present invention, the env coding sequence chosen will allow for the entry of the viral particle into the cells of the fertilized oocyte. In the methods of the present invention, the G-protein of vesicular-stomatitis virus (VSV-G) or a fragment or variant thereof is used in the env construct. Pseudotyping assays to determine additional envelope polypeptides useful in the methods of the present invention are well known in the art. It is further recognized that the trans-viral vector particle may be constructed in the absence of the Env construct.

A fragment or variant of an Envelope polypeptide will retain sufficient activity to support the production of a replication defective viral particle (i.e., capable of being incorporated into the envelope of a retroviral particle and capable of binding to target cells and allowing entry of the viral particle into the target cells). Assays for determining the function of an Env polypeptide or a fragment or variant thereof are known in the art. For example, expression of a fragment or variant of an Env polypeptide of the present invention will allow vector particles produced in that packaging cell line, to transmit a selectable marker to a naive sensitive cell such that it becomes resistant to the marker drug selection.

Any promoter sequence may be used in the env construct, so long as it is active in the packaging cell line. Such promoter sequences have been described elsewhere herein. Representative examples of suitable polyadenylation signals include the SV40 late polyadenylation signal, the bovine growth hormone termination/polyadenylation sequence, and the insulin polyadenylation signal.

The env construct may further comprise a nucleotide sequence encoding a selectable marker. Examples of such selectable markers include nucleotide sequences capable of conferring host resistance to antibiotics (e.g., puromycin, ampicillin, tetracycline, kanamycin, and the like), or conferring resistance to amino acid analogues, etc. Other selectable markers are well known in the art, including for example βgal, GFP, and luciferase. One of skill will appreciate the numerous possibilities.

As an illustrative and non-limiting example of the env construct of the present invention, is shown in FIG. 1. The construct comprises the CMV immediate early promoter operably linked to a VSV-G coding region operably linked to an SV40 polyadenylation signal.

5. Packaging Cell Lines

The trans-viral particles used in the methods of the present invention are generated using techniques known in the art. See, for example, U.S. Pat. No. 4,650,764, and U.S. patent application Ser. No. 09/089,900 herein incorporated by reference. The methods include incorporating into a packaging cell line the trans-enzyme construct, the gene transfer construct, the env construct, and the packaging construct; culturing the packaging cell line under suitable conditions that allow for the formation of viral particles; and, isolating the trans-viral particles. A wide variety of animal cells may be used to prepare the packaging cells of the present invention, including, for example, cells obtained from vertebrates, or mammals such as human, feline, goat, bovine, sheep, dog, and mice. Suitable packaging cell lines include, but are not limited to, HeLa (ATCC No. CCL2); HT1080 (ATCC No. CCL121); 293 (ATCC No. 1573); and the 293T cell line.

The various vector constructs may be introduced into the packaging cells through any means known in the art. Such means include, but are not limited to, electroporation, the use of liposomes, and CaPO₄ precipitation. See, for example, Ausabel et al. (1994) Current Protocols in Molecular Biology; John Wileg and Sons, Inc., and U.S. Pat. No. 5,739,081, both of which are herein incorporated by reference. It is further recognized that the various constructs of the trans-lentiviral system can be transiently expressed in the packaging cell line, or alternatively, any or all of the constructs can be stably incorporated into the genome of the packaging cell.

Methods for culturing the packaging cell line under conditions in which the trans-lentiviral particle is produced and the subsequent isolation of retroviral vector particles are also known in the art. For instance, the retroviral vector packaging cell lines may be cultured using standard culturing techniques, including any of a variety of monolayer culture systems. Such packaging cell lines may be cultured in T-flasks, roller bottles, or bioreactors. Any acceptable culture medium may be used, such as, AIM-V medium (Gibco BRL, Grand Island, N.Y.) containing 5% fetal bovine serum, or Dulbecco's modified Eagle medium (DMEM) with high glucose (4.5 g/l) supplemented with 10% heat-inactivated fetal bovine serum

Methods of isolating retroviral vector particles are known in the art, see for example, U.S. Pat. No. 5,661,022 and U.S. Pat. No. 6,013,517; herein incorporated by reference. As used herein, “purified trans-virus vector particles” means a preparation of trans-viral vector particles containing at least 50%, 60%, 70%, 80% by weight, preferably at least 85% by weight, and more preferably at least 90%, 93%, 95%, 98%, 99% by weight, of the retroviral vector particles.

III. Nucleotide Sequences of Interest

In the methods and compositions of the present invention, the modified proviral genome of the lentiviral vector comprises at least one nucleotide sequence of interest. A nucleic acid sequence of interest may be heterologous or homologous to the genetically modified animal. By heterologous is intended a nucleotide sequence that is not naturally found in the genome of the animal. By homologous is intended a nucleotide sequence that is found in the animal in nature and is integrated into the animal's genome at a location which differs from that of the naturally occurring sequence.

Any nucleotide sequence of interest can be used in the methods of the present invention. Of particular interest are nucleotide sequences, whose expression results in a detectable phenotype. These results can be achieved by directing the expression of heterologous products or an increased expression of endogenous products in the mammal. Alternatively, the results can be achieved by providing for a reduction of expression of one or more endogenous products, for instance, various enzymes or cofactors in the animal. One of skill in the art will appreciate that such nucleotide sequences of interest may encode a polypeptide, a ribozyme, or an antisense sequence.

Genes of interest are reflective of the commercial markets and the interests of those involved in the development of the genetically modified animal. General categories of the nucleotide sequences of interest include, for example, those sequence involved in the production of recombinant polypeptides in the milk of female mammals; the production of animal models for the study of disease; the production of animals with higher resistance to disease (e.g. diseases of the mammary gland such as mastitis); and the production of recombinant polypeptides in the blood, urine or other suitable body fluid or tissue of the animal. In addition, the nucleotide sequence of interest can be chosen as a means to advance studies of development, mutagenesis, teratogenesis, animal growth, reproductivity, and the efficacy of gene modifications designed to improve, for instance, meat/milk production or impart specific agronomic traits.

Non-limiting examples of nucleotide sequences of interest include sequence encoding milk proteins (i.e., lactoferrin, lysozyme, secreted immunoglobulins, lactalbumin, bile salt-stimulated lipase, etc.); serum proteins (i.e., albumin, immunoglobulins, Factor VIII, Factor IX, protein C, etc.); and, industrial enzymes (i.e., proteases, lipases, chitinases, and liginases from prokaryotic and eukaryotic sources). The recombinant DNA sequences include genomic and cDNA sequences encoding the recombinant polypeptide. In addition, the nucleotide sequences of interest may be selected for its role in various physiological processes, and consequently, is used to produce model animal systems for use in drug development to evaluate compound efficacy and toxicity (Liggitt et al. (1992) Xenobiotica, 22(9-10): 1043-1054).

In specific embodiments, the nucleotide sequences comprise a ribozyme. Ribozymes are enzymes comprised of ribonucleic acid (RNA) that conduct a variety of reactions involving RNA, including cleavage and ligation of polynucleotide strands. The specificity of ribozymes is determined by base pairing (hydrogen bonding) between the targeting domain of the ribozyme and the substrate RNA. Altering the nucleotide sequence of the targeting domain can modify this specificity. The catalytic domain of a ribozyme can also be changed in order to increase the activity or stability of the enzyme. Methods of designing ribozymes are known in the art. See, for example, U.S. Pat. Nos. 5,646,031; 5,646,020; and 5,639,655; all of which are herein incorporated by reference.

Alternatively, the nucleotide sequence may comprise an antisense nucleotide sequence. In this embodiment, the nucleotide sequence of interest comprises a nucleotide having a sequence complementary to the targeted sequence of the genetically modified animal. The nucleic acid targeted by the antisense sequence may be either DNA or RNA. The targeted nucleotide sequence will comprise a cellular gene whose expression is associated with a phenotype of interest. The site or sites within the targeted gene where the antisense interaction is to occur will be such as to modulate the expression of the polypeptide encoded by the targeted sequence (either at the level of transcription or translation), and thereby alter the phenotype of the resulting genetically modified animal. Preferred sites to target with the antisense oligonucleotide include the region encompassing the translation initiation or termination codon of the open reading frame (ORF) of the targeted gene. The transcription initiation site, or “5″ cap site” and the 5′ cap region (which encompasses from about 25 to about 50 contiguous nucleotides at the extreme 5′ terminus of a capped mRNA) may also be effective targets of the antisense nucleotide sequence.

In another embodiment, the nucleotide sequence of interest contained in the gene transfer vector comprises a polynucleotide sequence, which when expressed, comprises an RNA molecule capable of mediating RNA interference. By “RNA interference” is intended the phenomenon whereby the presence an RNA that is identical or has sufficient sequence identity to a target gene results in the degradation of messenger RNA transcribed from that target gene (Sharp (2001) Genes and Dev. 15: 485-490, herein incorporated by reference). In this embodiment, the viral vector is contacted with a target cell and the target cell (or organism) is maintained under conditions that allow for expression of the interfering RNA and consequently the degradation of the target mRNA.

An RNA that mediates RNA interference can comprise an RNA molecule(s), RNA segment(s) or RNA fragment(s). These terms include double-stranded RNA, single-stranded RNA, isolated RNA (partially purified RNA, essentially pure RNA, synthetic RNA, or recombinantly produced RNA), as well as altered RNA that differs from naturally occurring RNA by the addition, deletion, substitution and/or alteration of one or more nucleotides.

The interfering RNA expressed from the nucleotide sequence of interest used in this embodiment can comprise 2 or more nucleotides of a nucleic acid polymer per strand. In particular embodiments, the nucleotide sequence of interest comprising the interfering RNA is double stranded and comprises about 50 to about 40 nucleotide bases per strand, about 40 to about 30 nucleotide bases per strand or about 15 to about 50 nucleotide bases per strand. By “siRNA” is intended a short interfering RNA which is characterized as double-stranded RNA and is less than less than 30 nucleotide bases per strand or alternatively, about 10 to about 30 nucleotides per strand, about 15 to about 30 nucleotides per strand or about 21 to about 25 nucleotide bases per strand. It is recognized that it is not necessary that there be perfect correspondence of the interfering RNA sequence and the target sequence, but that the correspondence must be sufficient to enable the RNA to direct RNA interference cleavage of the target mRNA. Methods to assay for RNA interference are know in the art and include, for example, Northern analysis or assaying for a detectable phonotype. See, U.S. Application Publication 2002/0132788 and U.S. Application Publication 2002/0162126, both of which are herein incorporated by reference.

Methods of expressing and designing RNAs that mediate RNA interference are known in the art. See, for example, Paul et al. (2002) Nature Biotech. 20:505-508, herein incorporated by reference. For example, in one embodiment, the siRNAs are expressed as fold-back stem loop structures that give rise to siRNAs after intercellular processing. These siRNAs are processed intracellularly into siRNA-like molecules in the target cells. In another embodiment, the sense and antisense strands of the siRNAs are transcribed individually in the target cell. In this embodiment, a first siRNA comprising the sense strand of the target gene is expressed and a second siRNA comprising the antisense strand of the target gene is expressed. It is further recognized that the first and the second siRNA can be contained in the same or in separate gene transfer vectors. See, also, U.S. Application Publication No. 2002/0132788; U.S. Application Publication 2002/0162126 and U.S. Application No. 2002/0086356, all of which are herein incorporated by reference.

One of skill in the art will recognize various promoters can be used to express an interfering RNA. For example, the type III class of Pol III promoters, such as the U6 or H1 promoters, have proven successful. See, for example, Tuschl (2002) Nature Biotech. 20:446-448; Miyagishi and Taira (2002) Nature Biotech. 19:497-500; Paule and White (2000) Nucleic Acid Res. 28:1283-1298, all of which are herein incorporated by reference. Other promoters, such as PolII promoters, including, for example, the cytomegalovirus (CMV) promoter could be used. See, for example, Xia et al. (2002) Nature Biotechnology 20:1006-1010, herein incorporated by reference. One of skill will further recognize that a polyadenylation signal can also be added to improve expression of the interfering RNA.

It is further recognized that the interfering RNA can be used as a method for decreasing expression (partially or completely) of a targeted gene. This method of decreasing gene expression can be used therapeutically or for research (e.g., to generate models of disease states, to examine the function of a gene, to assess whether an agent acts on a gene, to validate targets for drug discovery, etc.). In those instances in which gene function is eliminated, the resulting cell or organism can also be referred to as a “knockout” or “knockdown”. In addition, many diseases arise from the abnormal expression of a particular gene or group of genes. RNA inteference could be used to inhibit the expression of the genes and thereby alleviate symptoms of or cure the disease.

The nucleotide sequence of interest is contained in a DNA construct. In specific embodiments, the DNA construct comprises all of the elements necessary for expression of a nucleotide sequence of interest in the target cell. Thus, the nucleotide sequence of interest contained in the modified proviral genome of the lentiviral vector, when introduced into the target cell via the viral particle, can be expressed.

By “operably linked” is intended the individual nucleotide sequences are joined such that expression of the nucleotide sequence of interest is under the regulatory control of the 5′ and 3′ regulatory sequences. When the nucleotide sequence of interest encodes a polypeptide, “operably linked” further encompasses the joining of the nucleotide sequences such that expression of the coding sequences occurs in the proper reading frame. The gene transfer vector may contain at least one additional nucleotide sequence of interest operably linked to a promoter.

As used herein, the DNA construct containing the nucleotide sequence of interest can include in the 5′-3′ direction of transcription, a transcriptional and translational initiation region, a nucleotide sequence of interest, and a transcriptional and translational termination region functional in the targeted host cell (i.e., a cell of the genetically modified animal). The transcriptional initiation region, the promoter, may be native or foreign to the target cell. Additionally, the promoter may be the natural sequence or, alternatively, a synthetic sequence. By “foreign” is intended that the transcriptional initiation region is not found in the target cell into which the lentiviral vector is introduced. While it may be preferable to express the sequences using heterologous promoters, the native promoter sequence may be used. The termination region may be native with the transcriptional initiation region, may be native with the operably linked DNA sequence of interest, or may be derived from another source.

Any promoter may be operably linked to the nucleotide sequence of interest so long as the promoter is active in the target cell. Such promoters may be constitutive promoters (i.e., Beta actin promoter (Balling et al. (1989) Cell 58:337-347 and Beddington et al. (1989) Development 105:733-737, the metallothionein promoter (Palmiter et al. (1983) Science 222:809-814 and Iwamoto et al. (1991) EMBO J. 10:3167-3175, the HMGCR promoter (Mehtali et al. (1990) Gene 91:179-184 and Tam et al. (1992) Development 115:703-715, and the histone H4 promoter (Choi et al (1991) Mol. Cell. Biol. 11:3070-3074). Additional promoter or enhancer elements can be found, for example, in the eukaryotic promoter database and in U.S. Pat. No. 6,271,436, herein incorporated by reference.

Alternatively, the promoter may be conditionally active. By a “conditional” promoter is intended the promoter is silent (or shows a reduce expression) until specifically activated. The promoter may be activated by an experimental manipulation, such as the administration of a drug or other activating agent, or alternatively, the promoter may be activated at a specific developmental stage or in a specific tissue.

Conditional promoters that are developmentally regulated include, for example, promoters active in undifferentiated cells including the phosphoglycerate kinase (Pgk) promoter and the octomer binding transcription factor 4 (Oct-4) promoter.

“Tissue preferred promoters” are expressed substantially only in the tissue or cell type chosen, although secondary expression in other tissue and/or cell types is acceptable if expression of the nucleotide sequence in the genetically modified animal in such tissue or cell type is not detrimental to the animal. Tissue-preferred promoters, include promoter which direct expression in milk, including but not limited to the whey acid promoter which directs expression in mammary tissues (European Patent Publication No. 0 264 166 and PCT Publication No. WO88/00239), the alpha lactalbumin promoter (PCT Publication No. WO88/01648), rat beta casein promoter (European Pat. Pub. No. 0 279 582) and the bovine casein promoter (PCT Pub. No. WO88/10118). See, also, for example, U.S. Pat. No. 5,741,957. Each of these references is herein incorporated by reference.

It is further recognized that the DNA construct comprising the nucleotide sequence of interest may contain various sequences that facilitate the expression, stabilization, and/or localization of the nucleotide sequence of interest and/or the resulting gene product. Such sequences include enhancers, introns, and post-transcriptional elements such as the Woodchuck Hepatitis Virus post-transcriptional region (WPRE) or PPT-CTS or functional variants thereof. See, for example, Zufferey et al. (1999) J. Virol. 73:2886-2892 and U.S. patent application Ser. No. 09/709,751, filed Nov. 10, 2000, both of which are herein incorporated by reference. In yet other embodiments the DNA construct comprising the nucleotide sequence of interest further includes affinity tags for purification or labeling (e.g., with antibodies).

Alternatively, the nucleotide sequence of interest may be contained in a DNA construct lacking a promoter sequence. In this embodiment, the nucleotide sequence of interest is expressed following integration of the sequence 5′ to an endogenous promoter of the target cell. One of skill in the art will be able to readily screen founder animals containing the nucleotide sequence of interest to identified animals which express the nucleotide sequence of interest in a desirable manner (i.e., the expression at the desired developmental stage, tissue, or level).

The proviral genome of the lentiviral vector may further contain at least one DNA construct comprising a selectable marker operably linked to a promoter. Selectable markers include, but are not limited to, luciferase (Lira et al. (1990) Proc. Natl. Acad. Sci. 87:7215-7219 and Lee et al. (1992) J. Biol. Chem. 267:15875-15885), β-gal (Goring et al. (1987) Science 235:456-458), GFP, chloramphenicol transferase (CAT) (Overbeek et al. (1985) Proc. Natl. Acad. Sci. 87:7815-7819), and the human growth hormone (hGH) (Pinkert et al. (1987) Genes Dev. 1:268-276). One of skill will appreciate that numerous possibilities exist.

One of skill will recognize the appropriate selectable marker to be used. For instance, lacZ is particularly useful for studies of tissue or position-specific gene expression in animals where whole embryos can be stained. CAT and luciferase are sensitive and quantitative, however, these markers do not allow visual detection for spatial pattern of expression. Human growth hormone can be easily detected and is useful for both spatial localization and quantitation.

In preparing the DNA construct, the various DNA fragments may be manipulated, so as to provide for the DNA sequences in the proper orientation and, as appropriate, in the proper reading frame. Toward this end, adapters or linkers may be employed to join the DNA fragments. For this purpose, in vitro mutagenesis, primer repair, restriction, annealing, resubstitutions, e.g., transitions and transversions, may be involved.

### EXPERIMENTAL

### Example 1

### Generating a Genetically Modified Mouse via Contacting a Lentiviral Vector with the Plasma Membrane

A. Trans-Lenti Viral Vector

Plasmids:

To construct the pPCW-eGFP gene transfer vector, a PCR amplified DNA fragment containing the EGFP cDNA (derived from pEGFP-C1, Clontech) was ligated into the BamHI/XhoI sites of the pHR-CMV-LacZ plasmid (Naldini et al. (1996) Science 272: 263-7), generating pHR-CMV-eGFP. Then, a 150 bp sequence of DNA (coordinates 4327 to 4483) containing the central polypurine tract (PPT) and central terminal site (CTS) was PCR-amplified from the HIV-1 pSG3 molecular clone (Ghosh et al. (1993) Virology 194: 858-864.) and ligated into the unique ClaI site of pHR-CMV-eGFP. To increase eGFP expression (Zufferey et al. (1999) J. Virol. 7:2886.), a post transcriptional regulator element derived from the woodchuck hepatitis virus (WPRE) was inserted down-stream of eGFP, generating the pPCW-eGFP gene transfer vector. This construct was used in the cell experiments set forth in Table 2, except with the Fisher 334(a) and Fisher 344(b) rats.

The transgenic animals reference in Table 2 as Fisher 334(a) were generated using a pgk-eGFP gene transfer vector. This construct is identical to pPCM-eGFP gene transfer vector described above except the phosphoglycerate kinase promoter (pgk) was used instead of the CMV promoter.

The transgenic animals reference in Table 2 as Fisher 344(b) were generated with a EF1alpha-APP-eGFP gene transfer vector. This construct represents a mutant version of Alzheimer's Amyloid Precursor protein (APP) linked to GFP through an IRES.

Preparation of Vector Stocks:

The trans-lentiviral vector represents an HIV-based vector with unique safety features that have been described earlier (Wu et al. (2000) Mol. Therapy 2:47-55.). Briefly, to reduce the risk of generating a replication competent retrovirus (RCR), the vector stocks were produced using the TranzVector™ (Tranzyme, Inc., Birmingham Ala.) lentiviral packaging system which separates the RT and 1N from Gag-Pol and delivers it in trans as a fusion partner with the HIV-1 virion associated protein Vpr. Thus, stocks of the trans-lentiviral vector were produced by transfecting 3 μg of the pCMV-gag-pro packaging plasmid, 1.0 μg of the pCMV-vpr-RT-IN trans-enzyme plasmid, 1.5 μg of the pMD.G (VSVG) expression plasmid, and 3 μg of the gene transfer (pPCW-eGFP) plasmid into subconfluent monolayer cultures of 293T cells by the calcium phosphate DNA precipitation method. Supernatants were harvested after 60 h, clarified by low-speed centrifugation (1000 g, 10 min) and filtered through 0.45-μm pore-size filters. The vector particles were concentrated by ultracentrifugation (Beckman SW28 rotor, 23,000 rpm, 2 hr). To determine vector titer, supernatant stock of 0.2, 0.04, 0.008, 0.0016, 0.00032, and 0.000064 μl were used to infect cultures of HeLa cells, and GFP-positive (green) cell colonies were counted 2 days later using a fluorescence microscope. Each GFP-positive cell colony was measured as a single transduction unit (TU). The titer of the purified virus used was 2.9×10⁹ TU/ml. Aliquots of virus were stored at −80° C. until use.

B. Isolation and Microinjection of Fertilized Mice Oocytes and Transfer of the Pre-Implementation Embryo

Female mice were super-ovulated by injections of PMS (pregnant mare serum containing FSH=follicle stimulating hormone) intraperitoneally (i.p.) at 50 IU per mouse. 46-48 hours later HCG (human gonadotrophin) was injected i.p. at 3.75 IU per mouse and mated.

The following day fertilized oocytes were harvested from the oviducts. Cumulus cells surrounding the harvested oocytes were removed by washing in hepes buffered embryo medium containing 1% hyaluronidase. After removal of cumulus cells, oocytes were washed in hepes buffered embryo medium removing residual hyaluronidase. Fertilized oocytes were transferred into ‘cleavage culture medium’ overlayed with paraffin oil. Oocytes were rested in a humidified incubator at 37.0° C., 7% CO₂ in air. After 0.5-4 hours oocytes were washed in hepes buffered embryo medium and then placed in a droplet onto a glass slide overlayed with paraffin oil. CVM-eGFP-transgene containing viral particles (at 10⁹/ml) were loaded into the glass injection needle and microinjected into the perivitelline space (PVS) of the oocytes. Injections were performed under 400× magnification. A volume of approximately 100 picoliters per oocyte was injected. Compared to pronuclear injections (resulting in oocyte lysis rates up to 33%) the oocyte lysis rate for PVS injection is around 5% or less.

Successfully injected oocytes were cultured overnight in ‘cleavage medium’ overlayed with paraffin oil at 37.0° C., 7% CO₂ in air. The next day oocytes have developed into two (2) cell embryos. Embryos were transferred into the oviducts of pseudo-pregnant females. After 19 days offspring were born. Observed pregnancy rate was 100% compared to 75% using pronuclear injection.

At 3 weeks of age injection derived progeny were screened by Southern blot analysis using an eGFP and a CMV diagnostic probe. Rate of genetically modified progeny obtained were 65% compared to ≦1% to 20% using pronuclear injection. Table 2 summaries the results of the present invention.

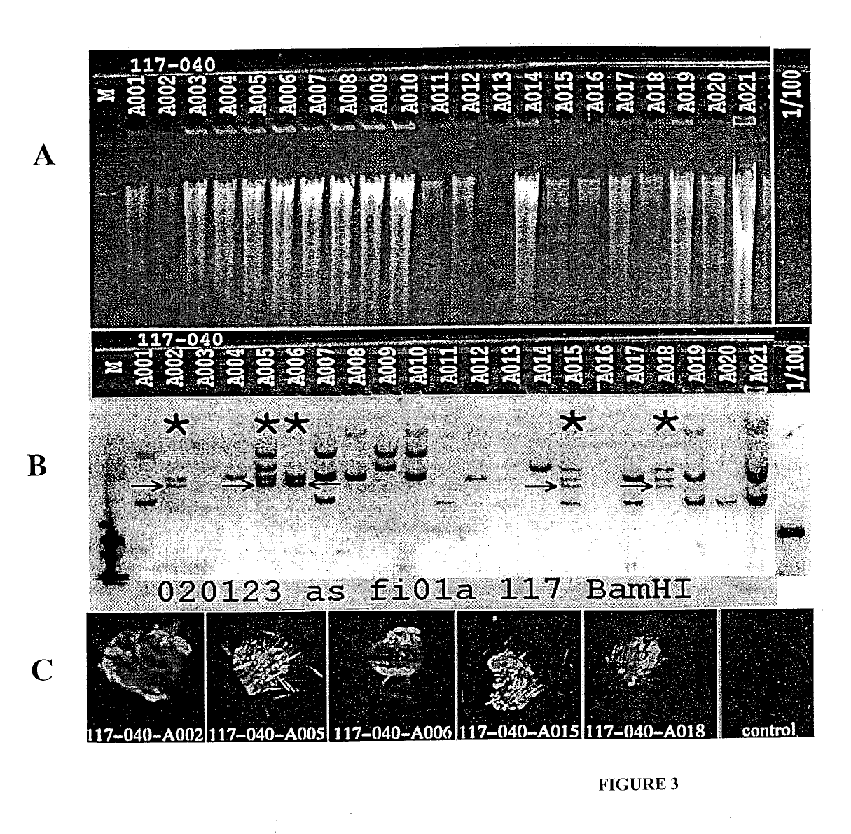

FIG. 3 provides evidence that the methods and compositions of the invention have the capacity to generate individual lines from each founder. FIG. 3A shows genomic DNA made from the tail tips of offspring (A generation) of one of the first eGFP transgenic founders 117_{—}040, which had been mated to a wild type mouse (BALB/cJ). The genomic DNA was restriction enzyme digested with BamHI, run on an 0.8% agarose gel (top image) and Southern blot transferred using standard protocols. The positive control on the gel is PeGFP-C2 (Clontech) restriction enzyme digested with AsnI,StuI and Alw44I and double gel purified.

FIG. 3B provides the Southern blot result following probing with ³²p labeled eGFP isolated from the PeGFP-C2 (Clontech) digested with Eco471II and EcoRI (˜750 bp) and double gel purified. The number of differing segregation patterns of the offspring seen in the southern image effectively demonstrates that the technology has the capacity to generate individual lines from each founder. In this case there are approximately 12 different independent integration patterns.

The arrows on the Southern blot autoradiograph (FIG. 3B) show the common banding pattern and therefore transgene integration of the mice expressing eGFP in the tail biopsies. The asterisk present in FIG. 3B represent the mice whose tails had then been biopsied (unfixed nor stained) and analyzed for eGFP fluorescence using a confocal microscope (BioRad Viewscan DVC 250 low laser power, 488 nm laser line under 4×objective). The images provided in FIG. 3C are confocal images showing definite fluorescence. The negative control for the confocal imaging was a negative litter mate genotyped A generation mouse.

TABLE 2
Efficiencies of TranzVector Transgenesis in Mouse and Rat Strains

| species | strain [1] | injected _{[2]} | transferred _{[3]} | pups _{[4]} | pups/trans [5] | genotyped _{[6]} | tg pups _{[7]} | tg/geno _{[8]} |
|---|---|---|---|---|---|---|---|---|
| mouse: | (FVB/nxBALB/cJ)F1 | 66 | 63 | 40 | 63.49% | 29 | 17 | 58.62% |
| | BALB/cJ | 292 | 192 | 14 | 7.29% | 14 | 8 | 57.14% |
| | C57BL/6 | 315 | 265 | 47 | 17.74% | 46 | 22 | 47.83% |
| | FVB | 272 | 194 | 34 | 17.53% | 34 | 25 | 73.53% |
| | NOD/Lt | 266 | 218 | 40 | 18.35% | 37 | 21 | 56.76% |
| | C3H/HeJ | 195 | 187 | 45 | 24.06% | 44 | 24 | 54.55% |
| | [9] total mouse | 1406 | 1119 | 220 | 19.66% | 204 | 117 | 57.35% |
| rat: | SD | 143 | 167 | 28 | 16.77% | 16 | 6 | 37.50% |
| | Wistar | 290 | 250 | 76 | 30.40% | 71 | 53 | 74.65% |
| | Fisher 344(a) | 302 | 245 | 27 | 11.02% | 26 | 19 | 70.37% |
| | Fisher 344(b) | 82 | 81 | 15 | 18.50% | 15- | 4 | 70.37% |
| | [10] total rat | 817 | 743 | 146 | 19.17% | 128- | 82 | 52.30% |
| | [11] TOTAL | 2223 | 1862 | 366 | 19.42% | 332 | 199 | 54.82% |

[1] The strain of mouse or rats used as an embryo donor;
_{[2]} The number of fertilized eggs injected into perivitelline space with TranzVector particles;
_{[3]} Number of embryos transferred into pseudo pregnant surrogate female mouse or rats;
_{[4]} Number pups born at the end of gestation from surrogate animals;
[5] Percentage pups born per transferred genetically manipulated embryos;
_{[6]} Number of born pups that were subsequently genotyped using Southern blot analysis;
_{[7]} Number of transgenic animals identified;
_{[8]} Percentage of transgenic animals per animals genotyped;
[9] Summary data for mouse experiments;
[10] Summary data for rat experiments;
[11] Total efficiency, mouse and rat combined

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

That which is claimed is:

1. A method of integrating a nucleotide sequence of interest into a genome of a vertebrate comprising:
a) providing an isolated early stage embryo having a plasma membrane; and,
b) contacting said plasma membrane with a composition comprising at least a first lentivirus vector comprising the nucleotide sequence of interest.

2. The method of claim 1, wherein
a) said isolated early stage embryo further comprises a zona pellucida, said zona pellucida and said plasma membrane defining a perivitelline space; and,
b) contacting said plasma membrane comprises introducing into said perivitelline space said composition.

3. The method of claim 1, wherein said early stage embryo is a fertilized ooctye.

4. The method of claim 1, wherein said early stage embryo is selected from the group consisting of a 2-cell stage embryo, a 4-cell stage embryo, an 8-cell stage embryo, and a morula.

5. The method of claim 2, wherein said composition comprising the first lentiviral vector is introduced into the perivitelline space via microinjection.

6. The method of claim 3, wherein said first nucleotide sequence of interest is integrated into the genome of the fertilized oocyte.

7. The method of claim 1, wherein said first nucleotide sequence of interest encodes a polypeptide.

8. The method of claim 1, wherein the first lentivirus vector is derived from a virus selected from the group consisting of a human immunodeficiency virus and a simian immunodeficiency virus.

9. The method of claim 1, wherein the first lentiviral vector is a trans-viral vector.

10. The method of claim 9, wherein the trans-viral vector is a trans-lentiviral vector or a trans-retroviral vector.

11. The method of claim 1, wherein the vertebrate is selected from the group consisting of mice, rabbits, sheep, cattle, aves, and rats.

12. The method of claim 1, wherein said composition further comprises a second lentivirus vector comprising a second nucleotide sequence of interest.

13. The method of claim 12, wherein the first and the second lentivirus vector is derived from a virus selected from the group consisting of a human immunodeficiency virus and a simian immunodeficiency virus.

14. The method of claim 12, wherein the first and the second lentiviral vector is a trans-viral vector.

15. The method of claim 14, wherein the trans-viral vector is a trans-lentiviral vector or a trans-retroviral vector.

16. The method of claim 1, wherein said method further comprises culturing the early stage embryo under conditions that allow formation of a pre-implantation embryo.

17. The method of claim 16, further comprising transferring said pre-implantation embryo into a recipient vertebrate and allowing said pre-implantation embryo to develop into at least one vertebrate.

18. The method of claim 16, wherein
a) said early stage embryo further comprises a zona pellucida, said zona pellucida and said plasma membrane defining a perivitelline space; and,
b) contacting said plasma membrane comprises introducing into said perivitelline space the composition comprising at least said first lentivirus vector comprising the first nucleotide sequence of interest.

19. The method of claim 16, wherein said early stage embryo is a fertilized oocyte.

20. The method of claim 16, wherein said early stage embryo is a 2-cell embryo, a 4-cell embryo, an 8-cell embryo, or a morula.

21. The method of claim 18, further comprising transferring said pre-implantation embryo into a recipient vertebrate and allowing said pre-implantation embryo to develop into at least one genetically modified vertebrate.

22. The method of claim 18, wherein said composition comprising the first lentiviral vector is introduced into the perivitelline space via microinjection.

23. The method of claim 19, wherein said first nucleotide sequence of interest is integrated into the genome of the fertilized oocyte.

24. The method of claim 16, wherein said composition further comprises a second lentivirus vector comprising a second nucleotide sequence of interest.

25. The method of claim 24, further comprising transferring said pre-implantation embryo to a recipient vertebrate and allowing said pre-implantation embryo to develop into at least one genetically modified vertebrate.

26. A composition comprising an isolated early stage embryo and an effective concentration of at least a first lentivirus vector comprising a nucleotide sequence of interest, wherein said early stage embryo is from a non-human vertebrate.

27. The composition of claim 26, wherein said early stage embryo is a fertilized oocyte.

28. The compositions of claim 26, wherein said early stage embryo is a 2-cell embryo, a 4-cell embryo, an 8-cell embryo, or a morula.

29. The composition of claim 26, wherein
a) said isolated early stage embryo further comprises a zona pellucida, said zona pellucida and said plasma membrane defining a perivitelline space; and,
b) said lentivirus vector is in the perivitelline space.

30. The composition of claim 29, wherein said first nucleotide sequence of interest encodes a polypeptide.

31. The composition of claim 26, wherein the lentivirus vector is derived from a virus selected from the group consisting of a human immunodeficiency virus and a simian immunodeficiency virus.

32. The composition of claim 26, wherein the lentiviral vector is a trans-viral vector.

33. The composition of claim 32, wherein the trans-viral vector is a trans-lentiviral vector or a trans-retroviral vector.

34. The composition of claim 26, wherein the non-human vertebrate is selected from the group consisting of mice, rabbits, sheep, cattle, aves, and rats.

35. The composition of claim 26, further comprising an effective concentration of a second lentivirus vector comprising a second nucleotide sequence of interest.

36. A method of integrating a nucleotide sequence of interest into the genome of a vertebrate comprising:
a) providing an isolated oocyte having a plasma membrane; and,
b) contacting said plasma membrane with a composition comprising at least a first lentivirus vector comprising the nucleotide sequence of interest.

37. The method of claim 36, wherein
a) said isolated oocyte further comprises a zona pellucida, said zona pellucida and said plasma membrane defining a perivitelline space; and,
b) contacting said plasma membrane comprises introducing into said perivitelline space said composition.

38. A composition comprising an isolated oocyte and an effective concentration of at least a first lentivirus vector comprising a nucleotide sequence of interest, wherein said oocyte is from a vertebrate.

39. The composition of claim 38, wherein
a) said isolated oocyte further comprises a zona pellucida, said zona pellucida and said plasma membrane defining a perivitelline space; and,
b) said lentivirus vector is in the perivitelline space.

40. A method of integrating a nucleotide sequence of interest into a genome of a vertebrate comprising:
a) providing an isolated blastula having a plasma membrane; and,
b) contacting said plasma membrane with a composition comprising at least a first lentivirus vector comprising the nucleotide sequence of interest.

41. The method of claim 40, wherein the lentivirus vector is derived from a virus selected from the group consisting of a human immunodeficiency virus and a simian immunodeficiency virus.

42. The method of claim 40, wherein the lentiviral vector is a trans-viral vector.

43. A composition comprising an isolated blastula and an effective concentration of at least a first lentivirus vector comprising a nucleotide sequence of interest, wherein said blastula is from a non-human vertebrate.
